SEQUENCE LISTING (203 sequences)

SEQ ID NO: 1 - protein, 10 aa, Homo sapiens
GFTFSSYVMN

SEQ ID NO: 2 - protein, 10 aa, Homo sapiens
GFTFRSHWLS

SEQ ID NO: 3 - protein, 10 aa, Homo sapiens
GFTFSSYVMN

SEQ ID NO: 4 - protein, 10 aa, Homo sapiens
GFTFRSHWLS

SEQ ID NO: 5 - protein, 10 aa, Homo sapiens
GFTFRSHWLS

SEQ ID NO: 6 - protein, 10 aa, Homo sapiens
GFTFRSHWLS

SEQ ID NO: 7 - protein, 10 aa, Homo sapiens
GFTFRSHWLS

SEQ ID NO: 8 - protein, 10 aa, Homo sapiens
GFTFRSHWLS

SEQ ID NO: 9 - protein, 10 aa, Homo sapiens
GFTFRSHWLS

SEQ ID NO: 10 - protein, 10 aa, Homo sapiens
GFTFRSHWLS

SEQ ID NO: 11 - protein, 10 aa, Homo sapiens
GFTFRSHWLS

SEQ ID NO: 12 - protein, 20 aa, Homo sapiens
WVSFISGDSSNTYYADSVKG

SEQ ID NO: 13 - protein, 20 aa, Homo sapiens
WVSNINYDGSSTYYADSVKG

SEQ ID NO: 14 - protein, 20 aa, Homo sapiens
WVSFISGDSSNTYYADSVKG

SEQ ID NO: 15 - protein, 20 aa, Homo sapiens
WVSNINYDGSSTYYADSVKG

SEQ ID NO: 16 - protein, 19 aa, Homo sapiens
WVSVTGVHGDTYYADSVKG

SEQ ID NO: 17 - protein, 19 aa, Homo sapiens
WVSVIGNWGDTYYADSVKG

SEQ ID NO: 18 - protein, 19 aa, Homo sapiens
WVSVTTHQGYTYYADSVKG

SEQ ID NO: 19 - protein, 19 aa, Homo sapiens
WVSATNRYGYTYYADSVKG

SEQ ID NO: 20 - protein, 20 aa, Homo sapiens
WVSNINYDGSSTYYADSVKG

SEQ ID NO: 21 - protein, 19 aa, Homo sapiens
WVSVITPYGDTYYADSVKG

SEQ ID NO: 22 - protein, 19 aa, Homo sapiens
WVSVITPYGDTYYADSVKG

SEQ ID NO: 23 - protein, 15 aa, Homo sapiens
TFMHGHLGGGLSMDF

SEQ ID NO: 24 - protein, 8 aa, Homo sapiens
DTYLHFDY

SEQ ID NO: 25 - protein, 15 aa, Homo sapiens
TFMHGHLGGGLSMDF

SEQ ID NO: 26 - protein, 8 aa, Homo sapiens
DTYLHFDY

SEQ ID NO: 27 - protein, 8 aa, Homo sapiens
DTYLHFDY

SEQ ID NO: 28 - protein, 8 aa, Homo sapiens
DTYLHFDY

SEQ ID NO: 29 - protein, 8 aa, Homo sapiens
DTYLHFDY

SEQ ID NO: 30 - protein, 8 aa, Homo sapiens
DTYLHFDY

SEQ ID NO: 31 - protein, 8 aa, Homo sapiens
DTYLHFDY

SEQ ID NO: 32 - protein, 8 aa, Homo sapiens
DTYLHFDY

SEQ ID NO: 33 - protein, 8 aa, Homo sapiens
DTYLHFDY

SEQ ID NO: 34 - protein, 11 aa, Homo sapiens
SGDNIGSFYVH

SEQ ID NO: 35 - protein, 20 aa, Homo sapiens
WVSNINYDGSSTYYADSVKG

SEQ ID NO: 36 - protein, 11 aa, Homo sapiens
SGDNIGSFYVH

SEQ ID NO: 37 - protein, 14 aa, Homo sapiens
TGTSSDVGDINDVS

SEQ ID NO: 38 - protein, 14 aa, Homo sapiens
TGTSSDVGDINDVS

SEQ ID NO: 39 - protein, 14 aa, Homo sapiens
TGTSSDVGDINDVS

SEQ ID NO: 40 - protein, 14 aa, Homo sapiens
TGTSSDVGDINDVS

SEQ ID NO: 41 - protein, 14 aa, Homo sapiens
TGTSSDVGDINDVS

SEQ ID NO: 42 - protein, 14 aa, Homo sapiens
TGTSSDVGDINDVS

SEQ ID NO: 43 - protein, 14 aa, Homo sapiens
TGTSSDVGDINDVS

SEQ ID NO: 44 - protein, 14 aa, Homo sapiens
TGTSSDVGDINDVS

SEQ ID NO: 45 - protein, 11 aa, Homo sapiens
LVIYDDNNRPS

SEQ ID NO: 46 - protein, 11 aa, Homo sapiens
LMIYDVNNRPS

SEQ ID NO: 47 - protein, 11 aa, Homo sapiens
LVIYDDNNRPS

SEQ ID NO: 48 - protein, 11 aa, Homo sapiens
LMIYDVNNRPS

SEQ ID NO: 49 - protein, 11 aa, Homo sapiens
LMIYDVNNRPS

SEQ ID NO: 50 - protein, 11 aa, Homo sapiens
LMIYDVNNRPS

SEQ ID NO: 51 - protein, 11 aa, Homo sapiens
LMIYDVNNRPS

SEQ ID NO: 52 - protein, 11 aa, Homo sapiens
LMIYDVNNRPS

SEQ ID NO: 53 - protein, 11 aa, Homo sapiens
LMIYDVNNRPS

SEQ ID NO: 54 - protein, 11 aa, Homo sapiens
LMIYDVNNRPS

SEQ ID NO: 55 - protein, 11 aa, Homo sapiens
LMIYDVNNRPS

SEQ ID NO: 56 - protein, 10 aa, Homo sapiens
GSWAGSSGSY

SEQ ID NO: 57 - protein, 10 aa, Homo sapiens
SSYGESLTSY

SEQ ID NO: 58 - protein, 10 aa, Homo sapiens
ASWTGVEPDY

SEQ ID NO: 59 - protein, 10 aa, Homo sapiens
QSYAGSYLSE

SEQ ID NO: 60 - protein, 10 aa, Homo sapiens
SSYGESLTSY

SEQ ID NO: 61 - protein, 10 aa, Homo sapiens
SSYGESLTSY

SEQ ID NO: 62 - protein, 10 aa, Homo sapiens
SSYGESLTSY

SEQ ID NO: 63 - protein, 10 aa, Homo sapiens
SSYGESLTSY

SEQ ID NO: 64 - protein, 10 aa, Homo sapiens
STYDGPGLSE

SEQ ID NO: 65 - protein, 10 aa, Homo sapiens
SSYGESLTSY

SEQ ID NO: 66 - protein, 10 aa, Homo sapiens
SSYGESLTSY

SEQ ID NO: 67 - protein, 124 aa, Homo sapiens
QVQLVESGGGLVQPGGSLRLSCAASGFTFSSYVMNWVRQAPGKGLEWVSFISGDSSNTYYADSVKGRFTISRDNSKNTLYLQMNSLRAEDTAVYYCARTFMHGHLGGGLSMDFWGQGTLVTVSS

SEQ ID NO: 68 - protein, 117 aa, Homo sapiens
QVQLVESGGGLVQPGGSLRLSCAASGFTFRSHWLSWVRQAPGKGLEWVSNINYDGSSTYYADSVKGRFTISRDNSKNTLYLQMNSLRAEDTAVYYCARDTYLHFDYWGQGTLVTVSS

SEQ ID NO: 69 - protein, 124 aa, Homo sapiens; identical to SEQ ID NO: 67

SEQ ID NO: 70 - protein, 117 aa, Homo sapiens; identical to SEQ ID NO: 68

SEQ ID NO: 71 - protein, 116 aa, Homo sapiens
QVQLVESGGGLVQPGGSLRLSCAASGFTFRSHWLSWVRQAPGKGLEWVSVTGVHGDTYYADSVKGRFTISRDNSKNTLYLQMNSLRAEDTAVYYCARDTYLHFDYWGQGTLVTVSS

SEQ ID NO: 72 - protein, 116 aa, Homo sapiens
QVQLVESGGGLVQPGGSLRLSCAASGFTFRSHWLSWVRQAPGKGLEWVSVIGNWGDTYYADSVKGRFTISRDNSKNTLYLQMNSLRAEDTAVYYCARDTYLHFDYWGQGTLVTVSS

SEQ ID NO: 73 - protein, 116 aa, Homo sapiens
QVQLVESGGGLVQPGGSLRLSCAASGFTFRSHWLSWVRQAPGKGLEWVSVTTHQGYTYYADSVKGRFTISRDNSKNTLYLQMNSLRAEDTAVYYCARDTYLHFDYWGQGTLVTVSS

SEQ ID NO: 74 - protein, 116 aa, Homo sapiens
QVQLVESGGGLVQPGGSLRLSCAASGFTFRSHWLSWVRQAPGKGLEWVSATNRYGYTYYADSVKGRFTISRDNSKNTLYLQMNSLRAEDTAVYYCARDTYLHFDYWGQGTLVTVSS

SEQ ID NO: 75 - protein, 117 aa, Homo sapiens; identical to SEQ ID NO: 68

SEQ ID NO: 76 - protein, 116 aa, Homo sapiens
QVQLVESGGGLVQPGGSLRLSCAASGFTFRSHWLSWVRQAPGKGLEWVSVITPYGDTYYADSVKGRFTISRDNSKNTLYLQMNSLRAEDTAVYYCARDTYLHFDYWGQGTLVTVSS

SEQ ID NO: 77 - protein, 116 aa, Homo sapiens; identical to SEQ ID NO: 76

SEQ ID NO: 78 - protein, 110 aa, Homo sapiens
DIELTQPPSVSVAPGQTARISCSGDNIGSFYVHWYQQKPGQAPVLVIYDDNNRPSGIPERFSGSNSGNTATLTISGTQAEDEADYYCGSWAGSSGSYVFGGRTKLTVLGQ

SEQ ID NO: 79 - protein, 113 aa, Homo sapiens
DIALTQPASVSGSPGQSITISCTGTSSDVGDINDVSWYQQHPGKAPKLMIYDVNNRPSGVSNRFSGSKSGNTASLTISGLQAEDEADYYCSSYGESLTSYVFGGGTKLTVLGQ

SEQ ID NO: 80 - protein, 110 aa, Homo sapiens
DIELTQPPSVSVAPGQTARISCSGDNIGSFYVHWYQQKPGQAPVLVIYDDNNRPSGIPERFSGSNSGNTATLTISGTQAEDEADYYCASWTGVEPDYVFGGGTKLTVLGQ

SEQ ID NO: 81 - protein, 111 aa, Homo sapiens
DIALTQPASVSGSPGQSITISCTGTSSDVGDINDVSWYQQHPGKAPKLMIYDVNNRPSGVSNRFSGSKSGNTASLTISGLQAEDEADYYCQSYAGSYLSEVFGGGTKLTVL

SEQ ID NO: 82 - protein, 113 aa, Homo sapiens; identical to SEQ ID NO: 79

SEQ ID NO: 83 - protein, 113 aa, Homo sapiens; identical to SEQ ID NO: 79

SEQ ID NO: 84 - protein, 113 aa, Homo sapiens; identical to SEQ ID NO: 79

SEQ ID NO: 85 - protein, 113 aa, Homo sapiens; identical to SEQ ID NO: 79

SEQ ID NO: 86 - protein, 113 aa, Homo sapiens
DIALTQPASVSGSPGQSITISCTGTSSDVGDINDVSWYQQHPGKAPKLMIYDVNNRPSGVSNRFSGSKSGNTASLTISGLQAEDEADYYCSTYDGPGLSEVFGGGTKLTVLGQ

SEQ ID NO: 87 - protein, 113 aa, Homo sapiens; identical to SEQ ID NO: 79

SEQ ID NO: 88 - protein, 113 aa, Homo sapiens; identical to SEQ ID NO: 86

SEQ ID NO: 89 - DNA, 372 bp, Homo sapiens
  source 1..372: mol_type = genomic DNA
caggtgcaattggtggaaagcggcggcggcctggtgcaaccgggcggcagcctgcgtctgagctgcgcggcctccggatttaccttttcttcttatgttatgaattgggtgcgccaagcccctgggaagggtctcgagtgggtgagctttatctctggtgattctagcaatacctattatgcggatagcgtgaaaggccgttttaccatttcacgtgataattcgaaaaacaccctgtatctgcaaatgaacagcctgcgtgcggaagatacggccgtgtattattgcgcgcgtacttttatgcatggtcatcttggtggtggtctttctatggatttttggggccaaggcaccctggtgacggttagctca

SEQ ID NO: 90 - DNA, 351 bp, Homo sapiens
  source 1..351: mol_type = genomic DNA
caggtgcaattggtggaaagcggcggcggcctggtgcaaccgggcggcagcctgcgtctgagctgcgcggcctccggatttacctttcgttctcattggctttcttgggtgcgccaagcccctgggaagggtctcgagtgggtgagcaatatcaattatgatggtagctctacctattatgcggatagcgtgaaaggccgttttaccatttcacgtgataattcgaaaaacaccctgtatctgcaaatgaacagcctgcgtgcggaagatacggccgtgtattattgcgcgcgtgatacttatcttcattttgattattggggccaaggcaccctggtgacggttagctca

SEQ ID NO: 91 - DNA, 372 bp, Homo sapiens; identical to SEQ ID NO: 89
  source 1..372: mol_type = genomic DNA

SEQ ID NO: 92 - DNA, 351 bp, Homo sapiens; identical to SEQ ID NO: 90
  source 1..351: mol_type = genomic DNA

SEQ ID NO: 93 - DNA, 348 bp, Homo sapiens
  source 1..348: mol_type = genomic DNA
caggtgcaattggtggaaagcggcggcggcctggtgcaaccgggcggcagcctgcgtctgagctgcgcggcctccggatttacctttcgttctcattggctttcttgggtgcgccaagcccctgggaagggtctcgagtgggtgagcgttactggtgttcatggtgatacttattatgctgattctgttaagggtcgttttaccatttcacgtgataattcgaaaaacaccctgtatctgcaaatgaacagcctgcgtgcggaagatacggccgtgtattattgcgcgcgtgatacttatcttcattttgattattggggccaaggcaccctggtgacggttagctca

SEQ ID NO: 94 - DNA, 348 bp, Homo sapiens
  source 1..348: mol_type = genomic DNA
caggtgcaattggtggaaagcggcggcggcctggtgcaaccgggcggcagcctgcgtctgagctgcgcggcctccggatttacctttcgttctcattggctttcttgggtgcgccaagcccctgggaagggtctcgagtgggtgagcgttattggtaattggggtgatacttattatgctgattctgttaagggtcgttttaccatttcacgtgataattcgaaaaacaccctgtatctgcaaatgaacagcctgcgtgcggaagatacggccgtgtattattgcgcgcgtgatacttatcttcattttgattattggggccaaggcaccctggtgacggttagctca

SEQ ID NO: 95 - DNA, 348 bp, Homo sapiens
  source 1..348: mol_type = genomic DNA
caggtgcaattggtggaaagcggcggcggcctggtgcaaccgggcggcagcctgcgtctgagctgcgcggcctccggatttacctttcgttctcattggctttcttgggtgcgccaagcccctgggaagggtctcgagtgggtgagcgttactactcatcagggttatacttattatgctgattctgttaagggtcgttttaccatttcacgtgataattcgaaaaacaccctgtatctgcaaatgaacagcctgcgtgcggaagatacggccgtgtattattgcgcgcgtgatacttatcttcattttgattattggggccaaggcaccctggtgacggttagctca

SEQ ID NO: 96 - DNA, 348 bp, Homo sapiens
  source 1..348: mol_type = genomic DNA
caggtgcaattggtggaaagcggcggcggcctggtgcaaccgggcggcagcctgcgtctgagctgcgcggcctccggatttacctttcgttctcattggctttcttgggtgcgccaagcccctgggaagggtctcgagtgggtgagcgctactaatcgttatggttatacttattatgctgattctgttaagggtcgttttaccatttcacgtgataattcgaaaaacaccctgtatctgcaaatgaacagcctgcgtgcggaagatacggccgtgtattattgcgcgcgtgatacttatcttcattttgattattggggccaaggcaccctggtgacggttagctca

SEQ ID NO: 97 - DNA, 351 bp, Homo sapiens; identical to SEQ ID NO: 90
  source 1..351: mol_type = genomic DNA

SEQ ID NO: 98 - DNA, 348 bp, Homo sapiens
  source 1..348: mol_type = genomic DNA
caggtgcaattggtggaaagcggcggcggcctggtgcaaccgggcggcagcctgcgtctgagctgcgcggcctccggatttacctttcgttctcattggctttcttgggtgcgccaagcccctgggaagggtctcgagtgggtgagcgttattactccttatggtgatacttattatgctgattctgttaagggtcgttttaccatttcacgtgataattcgaaaaacaccctgtatctgcaaatgaacagcctgcgtgcggaagatacggccgtgtattattgcgcgcgtgatacttatcttcattttgattattggggccaaggcaccctggtgacggttagctca

SEQ ID NO: 99 - DNA, 348 bp, Homo sapiens; identical to SEQ ID NO: 98
  source 1..348: mol_type = genomic DNA

SEQ ID NO: 100 - DNA, 330 bp, Homo sapiens
  source 1..330: mol_type = genomic DNA
gatatcgaactgacccagccgccttcagtgagcgttgcaccaggtcagaccgcgcgtatctcgtgtagcggcgataatattggttctttttatgttcattggtaccagcagaaacccgggcaggcgccagttcttgtgatttatgatgataataatcgtccctcaggcatcccggaacgctttagcggatccaacagcggcaacaccgcgaccctgaccattagcggcactcaggcggaagacgaagcggattattattgcggttcttgggctggttcttctggttcttatgtgtttggcggccgcacgaagttaaccgttcttggccag

SEQ ID NO: 101 - DNA, 339 bp, Homo sapiens
  source 1..339: mol_type = genomic DNA
gatatcgcactgacccagccagcttcagtgagcggctcaccaggtcagagcattaccatctcgtgtacgggtactagcagcgatgttggtgatattaatgatgtgtcttggtaccagcagcatcccgggaaggcgccgaaacttatgatttatgatgttaataatcgtccctcaggcgtgagcaaccgttttagcggatccaaaagcggcaacaccgcgagcctgaccattagcggcctgcaagcggaagacgaagcggattattattgctcttcttatggtgagtctcttacttcttatgtgtttggcggcggcacgaagttaaccgttcttggccag

SEQ ID NO: 102 - DNA, 330 bp, Homo sapiens
  source 1..330: mol_type = genomic DNA
gatatcgaactgacccagccgccttcagtgagcgttgcaccaggtcagaccgcgcgtatctcgtgtagcggcgataatattggttctttttatgttcattggtaccagcagaaacccgggcaggcgccagttcttgtgatttatgatgataataatcgtccctcaggcatcccggaacgctttagcggatccaacagcggcaacaccgcgaccctgaccattagcggcactcaggcggaagacgaagcggattattattgcgcttcttggactggtgttgagcctgattatgtgtttggcggcggcacgaagttaaccgttcttggccag

SEQ ID NO: 103 - DNA, 339 bp, Homo sapiens
  source 1..339: mol_type = genomic DNA
gatatcgcactgacccagccagcttcagtgagcggctcaccaggtcagagcattaccatctcgtgtacgggtactagcagcgatgttggtgatattaatgatgtgtcttggtaccagcagcatcccgggaaggcgccgaaacttatgatttatgatgttaataatcgtccctcaggcgtgagcaaccgttttagcggatccaaaagcggcaacaccgcgagcctgaccattagcggcctgcaagcggaagacgaagcggattattattgccagtcttatgctggttcttatctttctgaggtgtttggcggcggcacgaagttaaccgttcttggccag

SEQ ID NO: 104 - DNA, 339 bp, Homo sapiens; identical to SEQ ID NO: 101
  source 1..339: mol_type = genomic DNA

SEQ ID NO: 105 - DNA, 339 bp, Homo sapiens; identical to SEQ ID NO: 101
  source 1..339: mol_type = genomic DNA

SEQ ID NO: 106 - DNA, 339 bp, Homo sapiens; identical to SEQ ID NO: 101
  source 1..339: mol_type = genomic DNA

SEQ ID NO: 107 - DNA, 339 bp, Homo sapiens; identical to SEQ ID NO: 101
  source 1..339: mol_type = genomic DNA

SEQ ID NO: 108 - DNA, 339 bp, Homo sapiens
  source 1..339: mol_type = genomic DNA
gatatcgcactgacccagccagcttcagtgagcggctcaccaggtcagagcattaccatctcgtgtacgggtactagcagcgatgttggtgatattaatgatgtgtcttggtaccagcagcatcccgggaaggcgccgaaacttatgatttatgatgttaataatcgtccctcaggcgtgagcaaccgttttagcggatccaaaagcggcaacaccgcgagcctgaccattagcggcctgcaagcggaagacgaagcggattattattgctctacttatgatggtcctggtctttctgaggtgtttggcggcggcacgaagttaaccgttcttggccag

SEQ ID NO: 109 - DNA, 339 bp, Homo sapiens; identical to SEQ ID NO: 101
  source 1..339: mol_type = genomic DNA

SEQ ID NO: 110 - DNA, 339 bp, Homo sapiens; identical to SEQ ID NO: 108
  source 1..339: mol_type = genomic DNA

SEQ ID NO: 111 - protein, 469 aa, Homo sapiens
MAWVWTLPFLMAAAQSVQAQVQLVESGGGLVQPGGSLRLSCAASGFTFSSYVMNWVRQAPGKGLEWVSFISGDSSNTYYADSVKGRFTISRDNSKNTLYLQMNSLRAEDTAVYYCARTFMHGHLGGGLSMDFWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 112 - protein, 462 aa, Homo sapiens
MAWVWTLPFLMAAAQSVQAQVQLVESGGGLVQPGGSLRLSCAASGFTFRSHWLSWVRQAPGKGLEWVSNINYDGSSTYYADSVKGRFTISRDNSKNTLYLQMNSLRAEDTAVYYCARDTYLHFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 113 - protein, 469 aa, Homo sapiens; identical to SEQ ID NO: 111

SEQ ID NO: 114 - protein, 462 aa, Homo sapiens; identical to SEQ ID NO: 112

SEQ ID NO: 115 - protein, 461 aa, Homo sapiens
MAWVWTLPFLMAAAQSVQAQVQLVESGGGLVQPGGSLRLSCAASGFTFRSHWLSWVRQAPGKGLEWVSVTGVHGDTYYADSVKGRFTISRDNSKNTLYLQMNSLRAEDTAVYYCARDTYLHFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 116 - protein, 461 aa, Homo sapiens
MAWVWTLPFLMAAAQSVQAQVQLVESGGGLVQPGGSLRLSCAASGFTFRSHWLSWVRQAPGKGLEWVSVIGNWGDTYYADSVKGRFTISRDNSKNTLYLQMNSLRAEDTAVYYCARDTYLHFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 117 - protein, 461 aa, Homo sapiens
MAWVWTLPFLMAAAQSVQAQVQLVESGGGLVQPGGSLRLSCAASGFTFRSHWLSWVRQAPGKGLEWVSVTTHQGYTYYADSVKGRFTISRDNSKNTLYLQMNSLRAEDTAVYYCARDTYLHFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 118 - protein, 465 aa, Homo sapiens
MKHLWFFLLLVAAPRWVLSQVQLVESGGGLVQPGGSLRLSCAASGFTFRSHWLSWVRQAPGKGLEWVSATNRYGYTYYADSVKGRFTISRDNSKNTLYLQMNSLRAEDTAVYYCARDTYLHFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 119 - protein, 462 aa, Homo sapiens; identical to SEQ ID NO: 112

SEQ ID NO: 120 - protein, 461 aa, Homo sapiens
MAWVWTLPFLMAAAQSVQAQVQLVESGGGLVQPGGSLRLSCAASGFTFRSHWLSWVRQAPGKGLEWVSVITPYGDTYYADSVKGRFTISRDNSKNTLYLQMNSLRAEDTAVYYCARDTYLHFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 121 - protein, 461 aa, Homo sapiens; identical to SEQ ID NO: 120

SEQ ID NO: 122 - protein, 130 aa, Homo sapiens
MSVLTQVLALLLLWLTGTRCDIELTQPPSVSVAPGQTARISCSGDNIGSFYVHWYQQKPGQAPVLVIYDDNNRPSGIPERFSGSNSGNTATLTISGTQAEDEADYYCGSWAGSSGSYVFGGRTKLTVLGQ

SEQ ID NO: 123 - protein, 237 aa, Homo sapiens
MSVLTQVLALLLLWLTGTRCDIALTQPASVSGSPGQSITISCTGTSSDVGDINDVSWYQQHPGKAPKLMIYDVNNRPSGVSNRFSGSKSGNTASLTISGLQAEDEADYYCSSYGESLTSYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 124 - protein, 234 aa, Homo sapiens
MSVLTQVLALLLLWLTGTRCDIELTQPPSVSVAPGQTARISCSGDNIGSFYVHWYQQKPGQAPVLVIYDDNNRPSGIPERFSGSNSGNTATLTISGTQAEDEADYYCASWTGVEPDYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 125 - protein, 237 aa, Homo sapiens
MSVLTQVLALLLLWLTGTRCDIALTQPASVSGSPGQSITISCTGTSSDVGDINDVSWYQQHPGKAPKLMIYDVNNRPSGVSNRFSGSKSGNTASLTISGLQAEDEADYYCQSYAGSYLSEVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 126 - protein, 237 aa, Homo sapiens; identical to SEQ ID NO: 123

SEQ ID NO: 127 - protein, 237 aa, Homo sapiens; identical to SEQ ID NO: 123

SEQ ID NO: 128 - protein, 237 aa, Homo sapiens; identical to SEQ ID NO: 123

SEQ ID NO: 129 - protein, 237 aa, Homo sapiens; identical to SEQ ID NO: 123

SEQ ID NO: 130 - protein, 237 aa, Homo sapiens
MSVLTQVLALLLLWLTGTRCDIALTQPASVSGSPGQSITISCTGTSSDVGDINDVSWYQQHPGKAPKLMIYDVNNRPSGVSNRFSGSKSGNTASLTISGLQAEDEADYYCSTYDGPGLSEVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 131 - protein, 237 aa, Homo sapiens; identical to SEQ ID NO: 123

SEQ ID NO: 132 - protein, 237 aa, Homo sapiens; identical to SEQ ID NO: 130

SEQ ID NO: 133 - DNA, 1410 bp, Homo sapiens
  source 1..1410: mol_type = genomic DNA
atggcttgggtgtggaccttgccattcctgatggcagctgcccaaagcgtgcaggcccaggtgcagctggtcgagtctggcggcggactggtgcagcctggcggcagcctgagactgagctgcgccgccagcggcttcaccttcagcagctacgtgatgaactgggtgcggcaggcccctggcaagggcctggagtgggtgtccttcatcagcggcgacagcagcaacacctactacgccgacagcgtgaagggccggttcaccatcagccgggacaacagcaagaacaccctgtacctgcagatgaacagcctgcgggccgaggacaccgccgtgtactactgcgcccggaccttcatgcacggccacctgggcggaggactgagcatggatttctggggccagggcaccctggtcaccgtctcctcagcttccaccaagggcccatccgtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgacagtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 134 - DNA, 1389 bp, Homo sapiens
  source 1..1389: mol_type = genomic DNA
atggcttgggtgtggaccttgccattcctgatggcagctgcccaaggtgtccaggcccaggtgcagctggtcgagagcggcggagggctggtgcagcctggcggcagcctgagactgagctgcgccgccagcggcttcaccttcagaagccactggctgtcctgggtgcggcaggcccctggcaagggcctggaatgggtgtccaacatcaactacgacggcagcagcacctactacgccgacagcgtgaagggccggttcaccatcagccgggacaacagcaagaacaccctgtacctgcagatgaacagcctgcgggccgaggacaccgccgtgtactactgcgccagggacacctacctgcacttcgactactggggccagggcaccctggtcaccgtctcctcagcttccaccaagggcccatccgtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgacagtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 135 - DNA, 1410 bp, Homo sapiens; identical to SEQ ID NO: 133
  source 1..1410: mol_type = genomic DNA

SEQ ID NO: 136 - DNA, 1389 bp, Homo sapiens; identical to SEQ ID NO: 134
  source 1..1389: mol_type = genomic DNA

SEQ ID NO: 137 - DNA, 1386 bp, Homo sapiens
  source 1..1386: mol_type = genomic DNA
atggcttgggtgtggaccttgccattcctgatggcagctgcccaaagtgtccaggcccaggtgcagctggtcgagagcggcggagggctggtgcagcctggcggcagcctgagactgagctgcgccgccagcggcttcaccttcagaagccactggctgtcctgggtgcggcaggcccctggcaagggcctggaatgggtgtccgtgaccggcgtgcacggcgacacctactacgccgacagcgtgaagggccggttcaccatcagccgggacaacagcaagaacaccctgtacctgcagatgaacagcctgcgggccgaggacaccgccgtgtactactgcgccagggacacctacctgcacttcgactactggggccagggcaccctggtcaccgtctcctcagcttccaccaagggcccatccgtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgacagtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 138 - DNA, 1386 bp, Homo sapiens
  source 1..1386: mol_type = genomic DNA
atggcttgggtgtggaccttgccattcctgatggcagctgcccaaagtgtccaggcccaggtgcagctggtcgagagcggcggagggctggtgcagcctggcggcagcctgagactgagctgcgccgccagcggcttcaccttcagaagccactggctgtcctgggtgcggcaggcccctggcaagggcctggaatgggtgtccgtgatcggcaactggggcgacacctactacgccgacagcgtgaagggccggttcaccatcagccgggacaacagcaagaacaccctgtacctgcagatgaacagcctgcgggccgaggacaccgccgtgtactactgcgccagggacacctacctgcacttcgactactggggccagggcaccctggtcaccgtctcctcagcttccaccaagggcccatccgtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgacagtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 139 - DNA, 1386 bp, Homo sapiens
  source 1..1386: mol_type = genomic DNA
atggcttgggtgtggaccttgccattcctgatggcagctgcccaaagtgtccaggcccaggtgcagctggtcgagagcggcggagggctggtgcagcctggcggcagcctgagactgagctgcgccgccagcggcttcaccttcagaagccactggctgtcctgggtgcggcaggcccctggcaagggcctggaatgggtgtccgtgaccacccaccagggctacacctactacgccgacagcgtgaagggccggttcaccatcagccgggacaacagcaagaacaccctgtacctgcagatgaacagcctgcgggccgaggacaccgccgtgtactactgcgccagggacacctacctgcacttcgactactggggccagggcaccctggtcaccgtctcctcagcttccaccaagggcccatccgtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgacagtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 140 - DNA, 1386 bp, Homo sapiens
  source 1..1386: mol_type = genomic DNA
atggcttgggtgtggaccttgccattcctgatggcagctgcccaaagtgtccaggcccaggtgcagctggtcgagagcggcggagggctggtgcagcctggcggcagcctgagactgagctgcgccgccagcggcttcaccttcagaagccactggctgtcctgggtgcggcaggcccctggcaagggcctggaatgggtgtccgccaccaacagatacggctacacctactacgccgacagcgtgaagggccggttcaccatcagccgggacaacagcaagaacaccctgtacctgcagatgaacagcctgcgggccgaggacaccgccgtgtactactgcgccagggacacctacctgcacttcgactactggggccagggcaccctggtcaccgtctcctcagcttccaccaagggcccatccgtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgacagtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 141 - DNA, 1389 bp, Homo sapiens; identical to SEQ ID NO: 134
  source 1..1389: mol_type = genomic DNA

SEQ ID NO: 142 - DNA, 1386 bp, Homo sapiens
  source 1..1386: mol_type = genomic DNA
atggcttgggtgtggaccttgccattcctgatggcagctgcccaaagtgtccaggcccaggtgcagctggtcgagagcggcggagggctggtgcagcctggcggcagcctgagactgagctgcgccgccagcggcttcaccttcagaagccactggctgtcctgggtgcggcaggcccctggcaagggcctggaatgggtgtccgtgatcaccccctacggcgacacctactacgccgacagcgtgaagggccggttcaccatcagccgggacaacagcaagaacaccctgtacctgcagatgaacagcctgcgggccgaggacaccgccgtgtactactgcgccagggacacctacctgcacttcgactactggggccagggcaccctggtcaccgtctcctcagcttccaccaagggcccatccgtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgacagtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 143 - DNA, 1386 bp, Homo sapiens; identical to SEQ ID NO: 142
  source 1..1386: mol_type = genomic DNA

SEQ ID NO: 144 - DNA, 393 bp, Homo sapiens
  source 1..393: mol_type = genomic DNA
atgaaaaagacagctatcgcgattgcagtggcactggctggtttcgctaccgtagcgcaggccgatatcgaactgacccagccgccttcagtgagcgttgcaccaggtcagaccgcgcgtatctcgtgtagcggcgataatattggttctttttatgttcattggtaccagcagaaacccgggcaggcgccagttcttgtgatttatgatgataataatcgtccctcaggcatcccggaacgctttagcggatccaacagcggcaacaccgcgaccctgaccattagcggcactcaggcggaagacgaagcggattattattgcggttcttgggctggttcttctggttcttatgtgtttggcggccgcacgaagttaaccgttcttggccag

SEQ ID NO: 145 - DNA, 714 bp, Homo sapiens
  source 1..714: mol_type = genomic DNA
atgagtgtgctcactcaggtcctggcgttgctgctgctgtggcttacaggtacgcgttgcgacatcgccctgacccagcccgccagcgtgagcggcagccctggccagagcatcaccatcagctgcaccggcaccagcagcgacgtgggcgacatcaacgacgtgagctggtatcagcagcaccccggcaaggcccccaagctgatgatctacgacgtgaacaaccggcccagcggcgtgagcaaccggttcagcggcagcaagagcggcaacaccgccagcctgaccatcagcggcctccaggccgaggacgaggccgactactactgcagcagctacggcgagagcctgaccagctacgtgtttggcggcggaaccaagcttaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagacaaccacaccctccaaacaaagcaacaacaagtacgcggccagcagctatctgagcctgacgcctgagcagtggaagtcccacagaagctacagctgccaggtcacgcatgaagggagcaccgtggaaaagacagtggcccctacagaatgttcatag

SEQ ID NO: 146 - DNA, 705 bp, Homo sapiens
  source 1..705: mol_type = genomic DNA
atgagtgtgctcactcaggtcctggcgttgctgctgctgtggcttacaggtacgcgttgcgacatcgagctgacccagccccccagcgtgagcgtggcccctggccagaccgcccggatcagctgcagcggcgacaacatcggcagcttctacgtgcactggtatcagcagaagcccggccaggcccccgtgctggtgatctacgacgacaacaaccggcccagcggcatccccgagcggttcagcggcagcaacagcggcaacaccgccaccctgaccatcagcggcacccaggccgaggacgaggccgactactactgcgccagctggaccggcgtggagcccgactacgtgtttggcggcggaacaaagcttaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagacaaccacaccctccaaacaaagcaacaacaagtacgcggccagcagctatctgagcctgacgcctgagcagtggaagtcccacagaagctacagctgccaggtcacgcatgaagggagcaccgtggaaaagacagtggcccctacagaatgttcatag

SEQ ID NO: 147 - DNA, 714 bp, Homo sapiens
  source 1..714: mol_type = genomic DNA
atgagtgtgctcactcaggtcctggcgttgctgctgctgtggcttacaggtacgcgttgcgacatcgccctgacccagcccgccagcgtgagcggcagccctggccagagcatcaccatcagctgcaccggcaccagcagcgacgtgggcgacatcaacgacgtgagctggtatcagcagcaccccggcaaggcccccaagctgatgatctacgacgtgaacaaccggcccagcggcgtgagcaaccggttcagcggcagcaagagcggcaacaccgccagcctgaccatcagcggcctccaggccgaggacgaggccgactactactgccagagctacgccggcagctacctgagcgaggtgttcggcggagggaccaagcttaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagacaaccacaccctccaaacaaagcaacaacaagtacgcggccagcagctatctgagcctgacgcctgagcagtggaagtcccacagaagctacagctgccaggtcacgcatgaagggagcaccgtggaaaagacagtggcccctacagaatgttcatag

SEQ ID NO: 148 - DNA, 714 bp, Homo sapiens; identical to SEQ ID NO: 145
  source 1..714: mol_type = genomic DNA

SEQ ID NO: 149 - DNA, 714 bp, Homo sapiens; identical to SEQ ID NO: 145
  source 1..714: mol_type = genomic DNA

SEQ ID NO: 150 - DNA, 714 bp, Homo sapiens; identical to SEQ ID NO: 145
  source 1..714: mol_type = genomic DNA

SEQ ID NO: 151 - DNA, 714 bp, Homo sapiens; identical to SEQ ID NO: 145
  source 1..714: mol_type = genomic DNA

SEQ ID NO: 152 - DNA, 714 bp, Homo sapiens
  source 1..714: mol_type = genomic DNA
atgagtgtgctcactcaggtcctggcgttgctgctgctgtggcttacaggtacgcgttgcgacatcgccctgacccagcccgccagcgtgagcggcagccctggccagagcatcaccatcagctgcaccggcaccagcagcgacgtgggcgacatcaacgacgtgagctggtatcagcagcaccccggcaaggcccccaagctgatgatctacgacgtgaacaaccggcccagcggcgtgagcaaccggttcagcggcagcaagagcggcaacaccgccagcctgaccatcagcggcctccaggccgaggacgaggccgactactactgcagcacctacgacggccctggcctgagcgaggtgttcggcggagggaccaagcttaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagacaaccacaccctccaaacaaagcaacaacaagtacgcggccagcagctatctgagcctgacgcctgagcagtggaagtcccacagaagctacagctgccaggtcacgcatgaagggagcaccgtggaaaagacagtggcccctacagaatgttcatag

SEQ ID NO: 153 - DNA, 714 bp, Homo sapiens; identical to SEQ ID NO: 152
  source 1..714: mol_type = genomic DNA

SEQ ID NO: 154 - DNA, 714 bp, Homo sapiens; identical to SEQ ID NO: 152
  source 1..714: mol_type = genomic DNA

SEQ ID NO: 155 - protein, 213 aa, Homo sapiens
MQLPLALCLVCLLVHTAFRVVEGQGWQAFKNDATEIIPELGEYPEPPPELENNKTMNRAENGGRPPHHPFETKDVSEYSCRELHFTRYVTDGPCRSAKPVTELVCSGQCGPARLLPNAIGRGKWWRPSGPDFRCIPDRYRAQRVQLLCPGGEAPRARKVRLVASCKCKRLTRFHNQSELKDFGTEAARPQKGRKPRPRARSAKANQAELENAY

SEQ ID NO: 156 - protein, 14 aa, Homo sapiens
ARLLNAIGRGKWWR

SEQ ID NO: 157 - protein, 15 aa, Homo sapiens
RLVASCKCKRLTRFH

SEQ ID NO: 158 - protein, 13 aa, synthetic construct
  REGION 1..13: note = Artificial sequence
GGGSGGGGSGGGG

SEQ ID NO: 159 - protein, 19 aa, synthetic construct
  REGION 1..19: note = Artificial sequence
GGGGSGGGGSGGGGSGGGG

SEQ ID NO: 160 - DNA, 21 bp, synthetic construct
  misc_feature 1..21: note = Artificial sequence
  source 1..21: mol_type = other DNA
taaattatcataaagtcctaa

SEQ ID NO: 161 - DNA, 21 bp, synthetic construct
  misc_feature 1..21: note = Artificial sequence
  source 1..21: mol_type = other DNA
aggactttatgataatttatt

SEQ ID NO: 162 - DNA, 21 bp, synthetic construct
  misc_feature 1..21: note = Artificial sequence
  source 1..21: mol_type = other DNA
atagtggttaaataactccag

SEQ ID NO: 163 - DNA, 21 bp, synthetic construct
  misc_feature 1..21: note = Artificial sequence
  source 1..21: mol_type = other DNA
ggagttatttaaccactattt

SEQ ID NO: 164 - DNA, 21 bp, synthetic construct
  misc_feature 1..21: note = Artificial sequence
  source 1..21: mol_type = other DNA
taaattctcgtgatgtgccat

SEQ ID NO: 165 - DNA, 21 bp, synthetic construct
  misc_feature 1..21: note = Artificial sequence
  source 1..21: mol_type = other DNA
ggcacatcacgagaatttatt

SEQ ID NO: 166 - DNA, 21 bp, synthetic construct
  misc_feature 1..21: note = Artificial sequence
  source 1..21: mol_type = other DNA
tttcttatagcacagctggtt

SEQ ID NO: 167 - DNA, 21 bp, synthetic construct
  misc_feature 1..21: note = Artificial sequence
  source 1..21: mol_type = other DNA
ccagctgtgctataagaaatt

SEQ ID NO: 168 - DNA, 21 bp, synthetic construct
  misc_feature 1..21: note = Artificial sequence
  source 1..21: mol_type = other DNA
tagacctttccatccacgctg

SEQ ID NO: 169 - DNA, 21 bp, synthetic construct
  misc_feature 1..21: note = Artificial sequence
  source 1..21: mol_type = other DNA
gcgtggatggaaaggtctatt

SEQ ID NO: 170 - DNA, 29 bp, synthetic construct
  misc_feature 1..29: note = Artificial sequence
  source 1..29: mol_type = other DNA
atgcagctcccactggccctgtgtcttgt

SEQ ID NO: 171 - DNA, 30 bp, synthetic construct
  misc_feature 1..30: note = Artificial sequence
  source 1..30: mol_type = other DNA
aatcaggccgagctggagaacgcctactag

SEQ ID NO: 172 - protein, 443 aa, Homo sapiens
QVQLVESGGGLVQPGGSLRLSCAASGFTFRSHWLSWVRQAPGKGLEWVSNINYDGSSTYYADSVKGRFTISRDNSKNTLYLQMNSLRAEDTAVYYCARDTYLHFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 173 - protein, 217 aa, Homo sapiens
DIALTQPASVSGSPGQSITISCTGTSSDVGDINDVSWYQQHPGKAPKLMIYDVNNRPSGVSNRFSGSKSGNTASLTISGLQAEDEADYYCQSYAGSYLSEVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 174 - protein, 26 aa, Homo sapiens
CGPARLLPNAIGRGKWWRPSGPDFRC

SEQ ID NO: 175 - protein, 60 aa, Homo sapiens
DVSEYCRELHFTRSAKPVTELVCSGQCGPARWWRPSGPDFRCIPDRYRLVASCKCKRLTR

SEQ ID NO: 176 - protein, 133 aa, synthetic construct
  REGION 1..133: note = Synthetic polypeptide
  REGION 1..133: note = MISC_FEATURE - Humanized Antibody Sequence
MDTRAPTQLLGLLLLWLPGATFAQVLTQSPSSLSASVGDRVTITCQSSQSVYDNNWLAWYQQKPGKAPKLLIYDASDLASGVPSRFSGSGSGTDFTLTISSLQPEDFATYYCQGAYNDVIYAFGQGTKVEIKR

SEQ ID NO: 177 - protein, 131 aa, synthetic construct
  REGION 1..131: note = Synthetic polypeptide
  REGION 1..131: note = MISC_FEATURE - Humanized Antibody Sequence
METGLRWLLLVAVLKGVHCEVQLLESGGGLVQPGGSLRLSCAASGFSLSSYWMNWVRQAPGKGLEWVGTIDSGGRTDYASWAKGRFTISRDNSKNTMYLQMNSLRAEDTARYYCARNWNLWGQGTLVTVSS

SEQ ID NO: 178 - protein, 5 aa, Homo sapiens
SHWLS

SEQ ID NO: 179 - protein, 17 aa, Homo sapiens
NINYDGSSTYYADSVKG

SEQ ID NO: 180 - protein, 7 aa, Homo sapiens
DVNNRPS

SEQ ID NO: 181 - protein, 11 aa, Homo sapiens
QSYAGSYLSEV

SEQ ID NO: 182 - protein, 7 aa, Homo sapiens
GFTFRSH

SEQ ID NO: 183 - protein, 6 aa, Homo sapiens
NYDGSS

SEQ ID NO: 184 - protein, 8 aa, Homo sapiens
GFTFRSHW

SEQ ID NO: 185 - protein, 8 aa, Homo sapiens
INYDGSST

SEQ ID NO: 186 - protein, 10 aa, Homo sapiens
ARDTYLHFDY

SEQ ID NO: 187 - protein, 9 aa, Homo sapiens
SSDVGDIND

SEQ ID NO: 188 - skipped

SEQ ID NO: 189 - protein, 10 aa, Homo sapiens
NINYDGSSTY

SEQ ID NO: 190 - protein, 6 aa, Homo sapiens
RSHWLS

SEQ ID NO: 191 - protein, 13 aa, Homo sapiens
WVSNINYDGSSTY

SEQ ID NO: 192 - protein, 9 aa, Homo sapiens
ARDTYLHFD

SEQ ID NO: 193 - protein, 10 aa, Homo sapiens
VGDINDVSWY

SEQ ID NO: 194 - protein, 10 aa, Homo sapiens
LMIYDVNNRP

SEQ ID NO: 195 - protein, 113 aa, Homo sapiens
DIALTQPASVSGSPGQSITISCTGTSSDVGDINDVSWYQQHPGKAPKLMIYDVNNRPSGVSNRFSGSKSGNTASLTISGLQAEDEADYYCQSYAGSYLSEVFGGGTKLTVLGQ

SEQ ID NO: 196 - protein, 449 aa, synthetic construct
  REGION 1..449: note = Synthetic polypeptide
  REGION 1..449: note = MISC_FEATURE - Humanized Antibody Sequence
EVQLVQSGAEVKKPGASVKVSCKASGYTFTDYNMHWVRQAPGQGLEWMGEINPNSGGAGYNQKFKGRVTMTTDTSTSTAYMELRSLRSDDTAVYYCARLGYDDIYDDWYFDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 197 - protein, 214 aa, synthetic construct
  REGION 1..214: note = Synthetic Polypeptide
  REGION 1..214: note = MISC_FEATURE - Humanized Antibody Sequence
DIQMTQSPSSLSASVGDRVTITCRASQDISNYLNWYQQKPGKAPKLLIYYTSRLLSGVPSRFSGSGSGTDFTLTISSLQPEDFATYYCQQGDTLPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 198 - protein, 123 aa, synthetic construct [sequence omitted]

SEQ ID NO: 199 - protein, 110 aa, synthetic construct
  REGION 1..110: note = Synthetic Polypeptide
  REGION 1..110: note = MISC_FEATURE - Humanized Antibody Sequence
DIQMTQSPSSLSASVGDRVTITCRASQDISNYLNWYQQKPGKAPKLLIYYTSRLLSGVPSRFSGSGSGTDFTLTISSLQPEDFATYYCQQGDTLPYTFGGGTKVEIKRTV

SEQ ID NO: 200 - protein, 444 aa, synthetic construct
  REGION 1..444: note = Synthetic polypeptide
  REGION 1..444: note = MISC_FEATURE - Humanized Antibody Sequence
QVQLVQSGAEVKKPGASVKVSCKVSGFPIKDTFQHWVRQAPGKGLEWMGWSDPEIGDTEYASKFQGRVTMTEDTSTDTAYMELSSLRSEDTAVYYCATGDTTYKFDFWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 201 - protein, 214 aa, synthetic construct
  REGION 1..214: note = Synthetic Polypeptide
  REGION 1..214: note = MISC_FEATURE - Humanized Antibody Sequence
DIQMTQSPSSLSASVGDRVTITCKASQDVHTAVAWYQQKPGKAPKLLIYWASTRWTGVPSRFSGSGSGTDFTLTISSLQPEDFATYYCQQYSDYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 202 - protein, 118 aa, synthetic construct [sequence omitted]

SEQ ID NO: 203 - protein, 110 aa, synthetic construct
  REGION 1..110: note = Synthetic Polypeptide
  REGION 1..110: note = MISC_FEATURE - Humanized Antibody Sequence
DIQMTQSPSSLSASVGDRVTITCKASQDVHTAVAWYQQKPGKAPKLLIYWASTRWTGVPSRFSGSGSGTDFTLTISSLQPEDFATYYCQQYSDYPWTFGGGTKVEIKRTV

(19) United States
(12) Patent Application Publication
(10) Pub. No.: US 2025/0011407 A1
(43) Pub. Date: Jan. 9, 2025

(54) METHODS OF USING ANTI-SCLEROSTIN ANTIBODIES IN TREATMENT OF OSTEOGENESIS IMPERFECTA

(71) Applicants: Mereo Biopharma 3 Limited, London (GB); Ultragenyx Pharmaceutical Inc., Novato, CA (US)

(72) Inventors: Alastair MacKinnon, London (GB); Arun Mistry, London (GB); Emil Kakkis, San Rafael, CA (US); Michael S. Ominsky, Ann Arbor, MI (US)

(21) Appl. No.: 18/696,578

(22) PCT Filed: Sep. 29, 2022
(86) PCT No.: PCT/US2022/077259

§ 371 Date: Mar. 28, 2024

Related U.S. Application Data
(60) Provisional application No. 63/250,918, filed on Sep. 30, 2021.
(60) Provisional application No. 63/374,982, filed on Sep. 8, 2022.

(51) Int. Cl. C07K 16/22; A61K 39/00; A61P 19/08

(52) U.S. Cl. CPC C07K 16/22; A61P 19/08; A61K 2039/505; A61K 2039/545; C07K 2317/24

(57) ABSTRACT

Disclosed are methods and dosing regimens for treating a patient suffering from osteogenesis imperfecta, comprising administering to the patient a therapeutically effective amount of an anti-sclerostin antibody. The invention also provides an anti-sclerostin antibody for use in the treatment of osteogenesis imperfecta, comprising administering a therapeutically effective amount of the anti-sclerostin antibody each month according to certain dosing regimens.

### CROSS-REFERENCE TO RELATED APPLICATIONS

This application claims priority to, and the benefit of, U.S. Provisional Patent Application No. 63/250,918, filed on Sep. 30, 2021, and U.S. Provisional Patent Application No. 63/374,982, filed on Sep. 8, 2022, which are hereby incorporated by reference in their entirety.

### SEQUENCE LISTING

The instant application contains a sequence listing, which has been submitted in XML format via EFS-Web. The contents of the XML copy named “ULG-5006PC_122626-5006_Sequence Listing”, which was created on Sep. 22, 2022 and is 227,000 bytes in size, the contents of which are incorporated herein by reference in their entirety.

### FIELD OF INVENTION

This invention relates to antibodies and dosing regimens and their use as pharmaceutical compositions, more specifically to the chronic use of anti-sclerostin antibodies in the treatment of osteogenesis imperfecta.

### BACKGROUND

Osteogenesis imperfecta (OI) is a rare genetic disorder of the connective tissue characterized by bone fragility and reduced bone mass. OI comprises a group of inherited disorders which primarily, but not always, arise from mutations in the genes encoding type I collagen. About 85% of the cases are linked to mutations in one of the two genes encoding type I collagen (COL1A1 and COL1A2). Clinically, OI is characterized by fragile bones that fracture easily and without any trauma.

The clinical classification system divides OI into types I-V. Type I 01 patients usually suffer from a mild non-deforming disease that is often associated with a premature stop codon in COL1A1. This defect results in a reduced rate of type I collagen production and quantitatively less collagen in bone. Patients with type II 01 usually die during the perinatal period, as a result of respiratory failure from multiple severe fractures that include the rib cage. Types III and IV OI are often associated with glycine substitution in COL1A1 and COL1A2, which is a qualitative defect that prevents the 3 polypeptide chains of type I collagen to intertwine properly to form a normal triple alpha helical structure. Type III OI is the most severe form of OI in those affected children who survive infancy, whereas patients with type IV have mild to moderate bone deformities.

Therapeutic agents useful for treating bone-related diseases include anti-sclerostin antibodies. High affinity, neutralizing, fully human anti-sclerostin monoclonal antibodies (collectively “the human anti-sclerostin monoclonal antibody”) and their potent in vitro activity and in vivo activity are disclosed in e.g. U.S. Pat. Nos. 7,879,322, 8,246,953, and 8,486,661, which are hereby incorporated in their entirety by reference thereto. The treatment of OI using an anti-sclerostin antibody is disclosed in WO2018/115879A1 and WO2018/115880A1, which are hereby incorporated in their entirety by reference thereto. Additional anti-sclerostin antibodies include those described, for example, in WO2013/019954A1; U.S. Pat. Nos. 8,003,108, 7,592,429, and 8,017,120; and U.S. Patent Application Publication No. 20110044978A1; which are hereby incorporated in their entirety by reference thereto. Formulations of such antibodies are disclosed, for example, in WO2021/030179A1, which is hereby incorporated in its entirety by reference thereto.

Additional anti-sclerostin antibodies include those described, for example, in WO2008/115732A2, which is hereby incorporated in its entirety by reference thereto.

Additional anti-sclerostin antibodies include those described, for example, in U.S. Pat. No. 10,449,250, which is hereby incorporated in its entirety by reference thereto.

Additional anti-sclerostin antibodies include those described, for example, in WO2015/087187A1, which is hereby incorporated in its entirety by reference thereto.

There remains a need for further and improved treatment options for OI.

### BRIEF SUMMARY OF THE INVENTION

The results of the study reported in the examples surprisingly suggest that human OI patients can successfully be treated long-term with an anti-sclerostin antibody. These results showed that although the effect of anti-sclerostin antibody on bone turnover biomarkers peaks after the first month of therapy, and then attenuates, the effect on bone mineral density (BMD) shows continuous improvement (considerably beyond the attenuation of the biomarker response).

Thus, in one aspect, the present invention provides methods for the long-term or chronic treatment of osteogenesis imperfecta (OI) with an anti-sclerostin antibody.

In some embodiments, the instant disclosure provides a method for treating osteogenesis imperfecta (OI) in a human patient comprising administering to the human patient a therapeutically effective amount of an anti-sclerostin antibody each month for a period of at least 13 consecutive months.

In some embodiments, the anti-sclerostin antibody comprises (a) a VH polypeptide sequence having at least 90 percent sequence identity to the amino acid sequence set forth in SEQ ID NO: 70; and/or (b) a VL polypeptide sequence having at least 90 percent sequence identity to the amino acid sequence set forth in SEQ ID NO: 81. In one embodiment, the anti-sclerostin antibody comprises a VH polypeptide sequence comprising the amino acid sequence set forth in SEQ ID NO: 70 and a VL polypeptide sequence comprising the amino acid sequence set forth in SEQ ID NO: 81.

In some embodiments, the anti-sclerostin antibody comprises (a) a VH polypeptide sequence having at least 90 percent sequence identity to the amino acid sequence set forth in SEQ ID NO: 198; and/or (b) a VL polypeptide sequence having at least 90 percent sequence identity to the amino acid sequence set forth in SEQ ID NO: 199. In one embodiment, the anti-sclerostin antibody comprises a VH polypeptide sequence comprising the amino acid sequence set forth in SEQ ID NO: 198 and a VL polypeptide sequence comprising the amino acid sequence set forth in SEQ ID NO: 199.

In some embodiments, the anti-sclerostin antibody comprises (a) a VH polypeptide sequence having at least 90 percent sequence identity to the amino acid sequence set forth in SEQ ID NO: 202; and/or (b) a VL polypeptide sequence having at least 90 percent sequence identity to the amino acid sequence set forth in SEQ ID NO: 203. In one embodiment, the anti-sclerostin antibody comprises a VH polypeptide sequence comprising the amino acid sequence set forth in SEQ ID NO: 202 and a VL polypeptide sequence comprising the amino acid sequence set forth in SEQ ID NO: 203.

In some embodiments, the anti-sclerostin antibody comprises (a) a heavy chain variable region CDR1 comprising an amino acid sequence set forth in SEQ ID NO: 4; (b) a heavy chain variable region CDR2 comprising an amino acid sequence set forth in SEQ ID NO: 15; (c) a heavy chain variable region CDR3 comprising an amino acid sequence set forth in SEQ ID NO: 26; (d) a light chain variable region CDR1 comprising an amino acid sequence set forth in SEQ ID NO: 37; (e) a light chain variable region CDR2 comprising an amino acid sequence set forth in SEQ ID NO: 48; and (f) a light chain variable region CDR3 comprising an amino acid sequence set forth in SEQ ID NO: 59.

In some embodiments, the anti-sclerostin antibody comprises (a) a heavy chain variable region CDR1 comprising an amino acid sequence set forth in SEQ ID NO: 178; (b) a heavy chain variable region CDR2 comprising an amino acid sequence set forth in SEQ ID NO: 179; (c) a heavy chain variable region CDR3 comprising an amino acid sequence set forth in SEQ ID NO: 26; (d) a light chain variable region CDR1 comprising an amino acid sequence set forth in SEQ ID NO: 37; (e) a light chain variable region CDR2 comprising an amino acid sequence set forth in SEQ ID NO: 180; and (f) a light chain variable region CDR3 comprising an amino acid sequence set forth in SEQ ID NO: 181.

In some embodiments, the anti-sclerostin antibody comprises (a) a heavy chain variable region CDR1 comprising an amino acid sequence set forth in SEQ ID NO: 4; (b) a heavy chain variable region CDR2 comprising an amino acid sequence set forth in SEQ ID NO: 179; (c) a heavy chain variable region CDR3 comprising an amino acid sequence set forth in SEQ ID NO: 26; (d) a light chain variable region CDR1 comprising an amino acid sequence set forth in SEQ ID NO: 37; (e) a light chain variable region CDR2 comprising an amino acid sequence set forth in SEQ ID NO: 180; and (f) a light chain variable region CDR3 comprising an amino acid sequence set forth in SEQ ID NO: 181.

In some embodiments, the anti-sclerostin antibody is one described in U.S. Pat. Nos. 7,879,322, 8,246,953, 8,486,661, 8,003,108, 7,592,429, 8,017,120, or 10,449,250; International Patent Application Number WO2018/115879A1, WO2018/115880A1, WO2013/019954A1, WO2008/115732A2, or WO2015/087187A1; or U.S. Patent Application Publication No. 20110044978A1; which are hereby incorporated in their entirety by reference thereto.

In some embodiments, the anti-sclerostin antibody is selected from setrusumab, romosozumab, and blosozumab.

In some embodiments, a therapeutically effective amount of an anti-sclerostin antibody is administered to a patient each month, i.e., monthly, for a period of at least 13 consecutive months. In one embodiment, a therapeutically effective amount of an anti-sclerostin antibody is administered to a patient each month, i.e., monthly, for a period of at least 18 consecutive months. In another embodiment, a therapeutically effective amount of an anti-sclerostin antibody is administered to a patient each month, i.e., monthly, for a period of at least 24 consecutive months. In yet another embodiment, a therapeutically effective amount of an anti-sclerostin antibody is administered to a patient each month, i.e., monthly, for a period of at least 30 consecutive months. In yet another embodiment, the anti-sclerostin antibody is administered monthly for a period of at least 36 consecutive months. In yet another embodiment, the anti-sclerostin antibody is administered monthly for a period of up to 18 years.

In some embodiments, administration of a therapeutically effective amount of an anti-sclerostin antibody increases trabecular bone mineral density (BMD) after 12 months treatment of the human patient. In a related embodiment, administering the therapeutically effective amount of the anti-sclerostin antibody increases BMD of lumbar spine by 5% or more after 12 months of treatment of the human patient. BMD may be measured by dual-energy x-ray absorptiometry (DXA). In some embodiments, administration of a therapeutically effective amount of an anti-sclerostin antibody increases bone mineral density (BMD) of lumbar spine by 5% or more after 12 months treatment of the human patient.

In some embodiments, the chronic or long-term dosing regimens disclosed herein are effective in the treatment of any genetic disease of the bone which results in fractures or weakness that would benefit from chronic dosing, such as OI. In some embodiments, the OI is type I 01, type III 01 or type IV OI. In some embodiments, the human patient has one or more mutations in the COL1A1 and/or COL1A2 genes.

In some embodiments, the human patient is a paediatric patient. In some embodiments, the human patient is an adult patient. In some embodiments, the human patient is a child aged 0-17 year and the anti-sclerostin antibody is administered at a dose of 20-50 mg/kg.

In some embodiments, the anti-sclerostin antibody is administered monthly at a dose of 10-50 mg/kg. In one embodiment, the anti-sclerostin antibody is administered monthly at a dose selected from 10 mg/kg, 15 mg/kg, 20 mg/kg, 25 mg/kg, 30 mg/kg, 35 mg/kg, 40 mg/kg, 45 mg/kg, and 50 mg/kg. In another embodiment, the anti-sclerostin antibody is administered monthly at a dose of 10 mg/kg. In yet another embodiment, the anti-sclerostin antibody is administered monthly at a dose of 20 mg/kg. In yet another embodiment, the anti-sclerostin antibody is administered monthly at a dose of 40 mg/kg.

In some embodiments, the anti-sclerostin antibody is administered intravenously or subcutaneously. In one embodiment, the anti-sclerostin antibody is administered intravenously.

### BRIEF DESCRIPTION OF THE DRAWINGS

FIG. 1 shows the clinical study design.

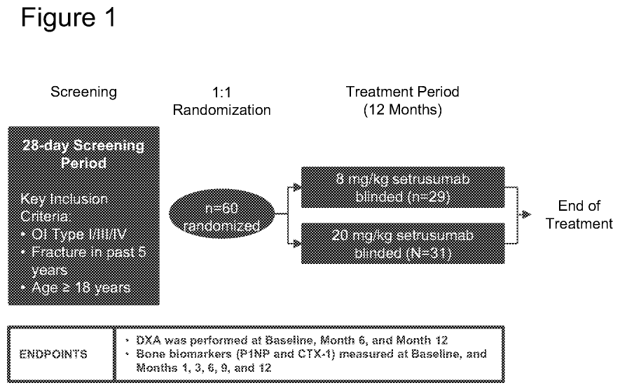

FIG. 2A and FIG. 2B show graphs depicting mean changes from baseline in serum levels of: FIG. 2A shows P1NP (procollagen 1 intact N-terminal propeptide), a bone formation biomarker; and FIG. 2B shows CTX-1 (C-terminal telopeptide), a bone resorption biomarker.

FIG. 3 shows a graph depicting mean change from baseline of BMD of lumbar spine data (measured by DXA), comparing monthly dosing with 8 mg/kg or 20 mg/kg setrusumab over a period of 12 months.

FIG. 4 shows graphs depicting effects of setrusumab discontinuation on BMD after 12 months with or without zoledronic acid therapy. FIG. 4A shows lumbar BMD after discontinuation. FIG. 4B shows total hip BMD after discontinuation. FIG. 4C shows radius total volumetric BMD after discontinuation. FIG. 4D shows tibia total volumetric BMD after discontinuation.

FIG. 5 shows graphs depicting effects of setrusumab discontinuation on bone turnover biomarkers after 12 months with or without zoledronic acid therapy. FIG. 5A shows serum P1NP levels after setrusumab discontinuation. FIG. 5B shows serum CTX-1 (i.e., “CTx”) levels after setrusumab discontinuation.

### DETAILED DESCRIPTION OF THE INVENTION

The present invention is based on the unexpected and surprising findings that, although biomarkers routinely used to determine bone turnover attenuate shortly after setrusumab therapy starts, setrusumab provides continuous increases in BMD in human patients with a bone-related disease (osteogenesis imperfecta; OI), which does not wane with the biomarkers. Human OI patients treated with monthly administrations of setrusumab for one year show continuous increase in BMD over the course of the treatment period: the increase in the first six months was similar to the increase in the second six months (Example 1). In contrast, the bone turnover biomarker response peaked after one month and then waned, returning to a level not statistically significantly different from baseline after six months of therapy. Whereas anti-sclerostin antibody-based therapies for other bone diseases have shown to lose efficacy over time and must be stopped after one year, the present findings show that long-term treatment with an anti-sclerostin antibody can provide continued efficacy for treatment of OI after 12 months of therapy.

By way of background, sclerostin is a naturally occurring protein that in humans is encoded by the SOST gene. Sclerostin is a secreted glycoprotein with a C terminal cysteine knot-like (CTCK) domain and sequence similarity to the DAN (differential screening-selected gene aberrative in neuroblastoma) family of bone morphogenetic protein (BMP) antagonists.

Anti-sclerostin antibodies have been shown to boost bone formation and density and provide beneficial effects in treating bone-related disorders in humans, including osteoporosis and OI. One anti-sclerostin antibody (romosozumab, marketed as EVENITY™) is approved by the FDA for treating osteoporosis in post-menopausal women at high risk for fracture. But the anabolic effect of EVENITY™ wanes after 12 monthly doses of therapy (see EVENITY™ prescribing information—“indications and usage” section, version revised 04/2020). So the duration of EVENITY™ use is limited to 12 monthly doses, after which continued therapy with an anti-resorptive agent is recommended if osteoporosis therapy remains warranted.

The present examples show that the effect of an anti-sclerostin antibody on BMD does not wane after 12 monthly doses for treatment of OI. This result is particularly surprising because the response of bone turnover biomarkers peaked after just one month of anti-sclerostin antibody therapy, after which the bone turnover biomarkers' response attenuated rapidly. The continued BMD increase after 12 months of therapy, even when the biomarker response waned considerably earlier, suggests that anti-sclerostin antibody treatment is particularly suitable for long-term therapy of OI. So monthly anti-sclerostin antibody therapy offers the advantage of prolonged treatment of OI, without needing to stop therapy because the antibody effect has waned. The clinical utility of anti-sclerostin antibody therapy therefore unexpectedly includes the chronic, or long-term, treatment of genetic diseases of the bone which result in fractures or weakness, such as OI, in humans.

As such, the invention is directed to methods of using an anti-sclerostin antibody in the chronic treatment of genetic diseases of the bone which result in fractures or weakness, such as OI. In one aspect the invention is concerned with the treatment of OI, with monthly administration of an anti-sclerostin antibody for at least 13 consecutive months.

The methods and uses of the anti-sclerostin antibody in the present invention were unexpected and surprising because the monthly treatment in OI patients results in continued increases in BMD over the course of 12 months, even though the response in bone turnover biomarkers peaks after one month and then attenuates.

### Definitions

In order that the present invention may be more readily understood, certain terms are first defined. Additional definitions are set forth throughout the detailed description.

The term “comprising” encompasses “including” as well as “consisting” e.g. a composition “comprising” X may consist exclusively of X or may include something additional e.g. X+Y.

The term “about” in relation to a numerical value x means, for example, x±10%.

The term “sclerostin” refers to human sclerostin as defined in SEQ ID NO: 155 (MQLPLALCLVCLLVHTAFRVVEGQGWQAFKNDATEIIPELGEYPEPPPELENNKTM NRAENGGRPPHHPFETKDVSEYSCRELHFTRYVTDGPCRSAKPVTELVCSGQCGPAR LLPNAIGRGKWWRPSGPDFRCIPDRYRAQRVQLLCPGGEAPRARKVRLVASCKCKR LTRFHNQSELKDFGTEAARPQKGRKPRPRARSAKANQAELENAY). Recombinant human sclerostin can be obtained from R&D Systems (Minneapolis, Minn., USA; 2006 cat #1406-ST-025). Additionally, recombinant mouse sclerostin/SOST is commercially available from R&D Systems (Minneapolis, Minn., USA; 2006 cat #1589-ST-025). U.S. Pat. Nos. 6,395,511 and 6,803,453, and U.S. Patent Publications 20040009535 and 20050106683 refer to anti-sclerostin antibodies in general.

The term “antibody” as used herein includes whole antibodies and any antigen binding fragment (i.e., “antigen-binding portion”) or single chains thereof. A naturally occurring “antibody” is a glycoprotein comprising at least two heavy (H) chains and two light (L) chains inter-connected by disulfide bonds. Each heavy chain comprises a heavy chain variable region (abbreviated herein as VH) and a heavy chain constant region. The heavy chain constant region comprises three domains, CH1, CH2 and CH3. Each light chain comprises a light chain variable region (abbreviated herein as VL) and a light chain constant region. The light chain constant region comprises one domain, CL. The VH and VL regions can be further subdivided into regions of hypervariability, termed complementarity determining regions (CDRs), interspersed with regions that are more conserved, termed framework regions (FRs). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. In one embodiment, reference to an antibody herein embraces isolated, monoclonal, human and humanized monoclonal antibodies.

The term “antigen-binding portion” of an antibody, as used herein, refers to full length or one or more fragments of an antibody that retain the ability to specifically bind to an antigen (e.g., sclerostin). It has been shown that the antigen-binding function of an antibody can be performed by fragments of a full-length antibody. Examples of binding fragments encompassed within the term “antigen-binding portion” of an antibody include a Fab fragment, a monovalent fragment consisting of the VL, VH, CL and CH1 domains; a F(ab)₂ fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., 1989 Nature 341:544-546), which consists of a VH domain; and an isolated complementarity determining region (CDR).

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., 1988 Science 242:423-426; and Huston et al., 1988 Proc. Natl. Acad. Sci. 85:5879-5883). Such single chain antibodies are also intended to be encompassed within the term “antigen-binding portion” of an antibody. These antibody fragments are obtained using conventional techniques known to those of skill in the art, and the fragments are screened for utility in the same manner as are intact antibodies.

An “isolated antibody”, as used herein, refers to an antibody that is substantially free of other antibodies having different antigenic specificities (e.g., an isolated antibody that specifically binds sclerostin is substantially free of antibodies that specifically bind antigens other than sclerostin). An isolated antibody that specifically binds sclerostin may, however, have cross-reactivity to other antigens, such as sclerostin molecules from other species. Moreover, an isolated antibody may be substantially free of other cellular material and/or chemicals. In one embodiment, reference to an antibody herein means an isolated antibody.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from sequences of human origin. Furthermore, if the human antibody contains a constant region, the constant region also is derived from such human sequences, e.g., human germline sequences, or mutated versions of human germline sequences, or from antibody containing consensus framework sequences derived from human framework sequences as described in Knappik, et al. (2000. J Mol Biol 296, 57-86).

The human antibodies may include amino acid residues not encoded by human sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In one embodiment, and as used herein, an antibody that binds sclerostin (e.g. an anti-sclerostin antibody) means that it specifically binds to sclerostin polypeptide. “Specifically binds to sclerostin polypeptide” is intended to refer to an antibody that binds to sclerostin polypeptide with a K_{D} of 1×10⁻⁸ M or less, 1×10⁻⁹ M or less, or 1×10⁻¹⁰ M or less. The term “K_{D}”, as used herein, is intended to refer to the dissociation constant, which is obtained from the ratio of K_{d} to Kₐ (i.e. K_{d}/Kₐ) and is expressed as a molar concentration (M). K_{D} values for antibodies can be determined using methods well established in the art. A method for determining the K_{D} of an antibody is by using surface plasmon resonance, or using a biosensor system such as a Biacore® system.

Standard assays to evaluate the binding ability of the antibodies toward sclerostin of various species are known in the art, including for example, ELISAs, western blots and RIAs. Suitable assays are described in detail in WO2009/047356. The binding kinetics (e.g., binding affinity) of the antibodies also can be assessed by standard assays known in the art, such as by Biacore analysis. Assays to evaluate the effects of the antibodies on functional properties of sclerostin (e.g., receptor binding, preventing or ameliorating osteolysis) are described in further detail in WO2009/047356.

As used herein, the percent identity between two sequences is a function of the number of identical positions shared by the sequences (i.e., % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol, Biol. 48:444-453, 1970) algorithm which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the BLASTX program (version 2.0) of Altschul, et al., 1990 J. Mol. Biol. 215:403-10. BLAST protein searches can be performed with the BLASTX program, score=50, wordlength=3 to obtain amino acid sequences homologous to the antibody molecules of the invention. To obtain gapped alignments for comparison purposes, Gapped BLAST can be utilized as described in Altschul et al., 1997 Nucleic Acids Res. 25(17):3389-3402. When utilizing BLAST and Gapped BLAST programs, the default parameters of the respective programs (e.g., BLASTX and BLASTN) can be used. See www.ncbi.nlm.nih.gov.

The terms “cross-block”, “cross-blocked” and “cross-blocking” are used interchangeably herein to mean the ability of an antibody or other binding agent to interfere with the binding of other antibodies or binding agents to sclerostin in a standard competitive binding assay. The ability or extent to which an antibody or other binding agent is able to interfere with the binding of another antibody or binding molecule to sclerostin, and therefore whether it can be said to cross-block according to the invention, can be determined using standard competition binding assays. One suitable assay involves the use of the Biacore technology (e.g. by using the Biacore 3000 instrument (Biacore, Uppsala, Sweden)), which can measure the extent of interactions using surface plasmon resonance technology. Another assay for measuring cross-blocking uses an ELISA-based approach. Further details on both methods are given in WO2009/047356, and are specifically incorporated herein by reference.

As used herein, “monthly” administration, or administration “each month”, refers to administration of a dose of anti-sclerostin antibody once per month for a given period of consecutive months.

Various aspects of the invention are described in further detail in the following subsections.

### Osteogenesis Imperfecta

The results of the examples surprisingly showed that anti-sclerostin therapy provides continuous increases in BMD in OI patients, long after the effect on bone turnover biomarkers wanes, or attenuates. Anti-sclerostin antibody therapy is therefore surprisingly suitable for long-term, or chronic, therapy of bone-related genetic diseases that result in bone weakness or increased incidence of bone fracture, such as OI.

In one aspect, the invention is directed to methods of treating patients with a bone-related genetic disease such as OI. The term patients, as used herein, means human patients.

In one aspect the invention provides use of an anti-sclerostin antibody for the manufacture of a medicament for the treatment of osteogenesis imperfecta. All of the other aspects/embodiments described herein apply equally to this particular aspect of the invention.

In another aspect the invention provides an anti-sclerostin antibody for use in the treatment of osteogenesis imperfecta. All of the other aspects/embodiments described herein apply equally to this particular aspect of the invention.

The examples report a study of anti-sclerostin antibody therapy in OI patients, and show that OI patients respond to monthly anti-sclerostin antibody therapy. Accordingly, in one embodiment, the methods and uses described herein are for treating osteogenesis imperfecta (OI) using anti-sclerostin antibodies described herein.

OI is classified by the genetics and severity of disease, and can be classified as type I OI, type II 01, type III 01, type IV OI or type V OI according to the classification of Van Dijk and Sillence (2014, Am J Med Genet Part A 164A:1470-1481 and Van Dijk and Sillence, 2014, Am J Med Genet Part A 167A:1178; which are hereby incorporated in their entirety by reference thereto). Classification relies on a combination of clinical evaluation/diagnosis, biochemical analysis as well as molecular genetic testing, and is routine for those skilled in the art. The OI nomenclature/classification as used herein is as proposed by Van Dijk and Sillence, as referenced in the publications above. The study participants had type I, type III, or type IV OI, and all OI types responded to setrusumab therapy. Accordingly, in a certain embodiment, the bone-related disease is type I 01, type III 01, or type IV OI.

In 80%-90% of people with OI, OI is caused by mutations in the COL1A1 and COL1A2 genes (17q21.33 and 7q22.3, respectively) encoding the alpha1 and alpha 2 chains of type-I collagen. A comprehensive database of over 1000 known mutations has been published along with a genotype-phenotype correlation (oi.gene.le.ac.uk/home.php; accessed 29 Sep. 2021). Mutations in other genes, such as CRTAP, LEPRE1 or PPIB, are also known. Molecular genetic tests for mutations in the COL1A1 and COL1A2 genes are known and routine for those skilled in the art. By way of example, Korkko et al. (1998) describe PCR amplification of the COL1A1 gene and the COL1A2 genes followed by mutation scanning by conformation-sensitive gel electrophoresis (CSGE) (Am. J. Hum. Genet. 62:98-110, 1998). van Dijk et al. (2010) describe COL1A1 mutation detection by a multiplex ligation-dependent probe amplification (MHLPA) technique (Genet Med 12(11):736-741). More recently, Árvai, K. et al. (2016) describe next-generation sequencing methods (Sci. Rep. 6, 28417). These references are hereby incorporated by reference thereto.

Accordingly, in one embodiment, the methods and uses described herein are for treating patients who exhibit a deficiency of type-I collagen, e.g. OI types I-IV. As a result, the normal architecture of bone, consisting of collagen fibrils and hydroxyapatite crystals, is altered and causes brittleness. In one embodiment, the methods and uses herein are for treating human OI patients characterized by one or more mutations in COL1A1 and/or COL1A2.

In one embodiment, the methods and uses described herein are for treating OI type I, III and/or IV. In one embodiment, OI type I, III and IV are confirmed by DNA testing i.e. detection of COL1A1/COL1A2 mutations. Thus, in one embodiment the methods and uses herein are for treating OI type I, III and/or IV characterized by one or more mutations in COL1A1 and/or COL1A2.

In some embodiments, the methods and uses of the anti-sclerostin antibody are for treating a mild to moderate form of OI. In other embodiments of the methods and uses of the anti-sclerostin antibody, the patient under treatment has a type I 01, a type II 01, a type III 01, or a type IV OI.

In a specific embodiment, the invention provides a method for treating OI in a human patient comprising administering to the human patient a therapeutically effective amount of setrusumab at a dose of 300-1500 mg each month for a period of at least 30 consecutive months up to 18 years.

### Paediatric Dosing

In some embodiments of the methods and uses of the anti-sclerostin antibody, the OI patients are adult patients aged 18 and above. In yet still other embodiments of the methods and uses of the anti-sclerostin antibody, the OI patients are pediatric patients. Pediatric patients as defined herein embraces children aged 0-17 such as those aged 2-17, 3-17, 4-17 or 5-17.

The rate of drug clearance compared to body weight or body surface area is higher in children (particularly small children) than it is in adults and therefore pediatric doses when expressed as, for example mg/kg, generally have to be increased, relative to the equivalent adult dose, to ensure that children receive sufficiently high doses of a drug to be efficacious. A suitable pediatric dose can be estimated from the ratio of the weights of a child and an adult to the power 0.7 (Pediatric Pharmacology—Therapeutic Principles in Practice, 2^{nd} Ed., Yaffe and Aranda), shown in Equation 1.

$$Pediatric dose conversion$$

$${dose}_{child} = {dose}_{adult} {( \frac{{weight}_{child}}{w e i g h {t}_{adult}} )}^{0.7} \qquad Equation 1$$

Applying this formula, a dose of 20 mg/kg in an adult patient (of 70 kg) equates to a dose of 40 mg/kg in a 7 kg child (˜6 month old). So the smallest patients may require a dose of up to 40 mg/kg setrusumab to achieve a therapeutic outcome similar to an adult dosed at 20 mg/kg. Higher mg/kg doses in adults would thus require still higher doses in paediatric patients. Accordingly, in one embodiment the patient is aged 0-17 years and the anti-sclerostin antibody is administered at a dose of 20-50 mg/kg each month. In a certain embodiment, the patient is aged 0-17 years and the anti-sclerostin antibody is administered at a dose of 20-40 mg/kg each month.

Thus, in a specific embodiment, the invention provides a method for treating OI in a human patient aged 0-17 years comprising administering to the human patient a therapeutically effective amount of an anti-sclerostin antibody at a dose of 20-50 mg/kg each month for a period of at least 13 consecutive months, e.g., at least 18 consecutive months, at least 24 consecutive months, or at least 30 consecutive months, up to 18 years.

Thus, in a specific embodiment, the invention provides a method for treating OI in a human patient aged 0-17 years comprising administering to the human patient a therapeutically effective amount anti-sclerostin antibody at a dose of 20-50 mg/kg each month for a period of at least 13 consecutive months, e.g., at least 18 consecutive months, at least 24 consecutive months, or at least 30 consecutive months, up to 18 years.

Throughout the present specification, numerical ranges are provided for certain quantities such as dosage amounts of an anti-sclerostin antibody. It is to be understood that these ranges comprise endpoints and all subranges therein, including each integer in and between a disclosed range. Thus, the range “from 20 to 50” includes all possible ranges therein (e.g., 21-49, 22-48, 23-47, etc.) as well as each individual integer from 20 to 50 (e.g., 20, 21, 22, 23, 24, etc.). Where ranges are provided in the form of fractions, percentages, decimals, and the like, such ranges likewise include all possible subranges therein and each individual fraction, percentage, decimal, etc. in and between the disclosed range. For example, the range “from 0.1 to 1.0” includes all possible ranges therein (e.g., 0.2 to 0.9, etc.) and each individual 1/10th decimal from 0.1 to 1.0 (e.g., 0.1, 0.2, 0.3, 0.4, etc.). Accordingly, dosage amounts of an anti-sclerostin antibody of 20-50 mg/kg include dosage amounts of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50 mg/kg of the anti-sclerostin antibody.

### Dosing Regimen

The invention is based on the discovery that anti-sclerostin antibody therapy can be used long-term, even though bone turnover biomarkers attenuate soon after therapy starts. The invention therefore relates to monthly administration of anti-sclerostin antibodies for at least 13 months. Dosing regimens are adjusted to provide the optimum desired response (e.g., a therapeutic response). For example, the dose may be reduced or increased as indicated by the exigencies of the therapeutic situation.

For administration of the anti-sclerostin antibody, the dosage ranges from about 8 milligram of said antibody per kilogram body weight of the patient (herein referred to as “mg/kg” throughout this application) to 50 mg/kg, more usually about 20 to 40 mg/kg. For example dosages can be about 20 mg/kg body weight, about 30 mg/kg body weight, about 40 mg/kg body weight, or about 50 mg/kg body weight. In another embodiment, the monthly dose is about 20 to 30 mg/kg.

In one embodiment, the anti-sclerostin antibody is administered at a dose of 20 mg per kg body weight of a patient, or at 21, 22, 23, 24, 25, 26, 27, 28, 29, 30, 31, 32, 33, 34, 35, 36, 37, 38, 39, or 40 mg/kg.

Table 2 reports that the weights of patients in the study of the present examples ranged from 19.9-120.7 kg. So, in a related embodiment, the anti-sclerostin antibody is administered at a dose of 150-3000 mg, for example 150-2500 mg. In one embodiment, the anti-sclerostin antibody is administered at a dose of 200-2500 mg, 400-2500 mg, 150-2000 mg, 200-2000 mg, 400-2000 mg, 150-1500 mg, 200-1500 mg, 400-1500 mg. In one embodiment, the anti-sclerostin antibody is administered at a dose of 200-2500 mg. In one embodiment, the anti-sclerostin antibody is administered at a dose of 400-2500 mg. In one embodiment, the anti-sclerostin antibody is administered at a dose of 150-2000 mg. In one embodiment, the anti-sclerostin antibody is administered at a dose of 200-2000 mg. In one embodiment, the anti-sclerostin antibody is administered at a dose of 400-2000 mg. In one embodiment, the anti-sclerostin antibody is administered at a dose of 150-1500 mg. In one embodiment, the anti-sclerostin antibody is administered at a dose of 200-1500 mg. In one embodiment, the anti-sclerostin antibody is administered at a dose of 400-1500 mg.

In one embodiment, the anti-sclerostin antibody is administered at a dose of 200 mg, or 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, or 2450 mg.

The invention relates to the surprising suitability of anti-sclerostin antibody for long-term therapy. According to the method of the invention, the anti-sclerostin antibody is therefore administered monthly for a period of at least 13 consecutive months. In some embodiments, the anti-sclerostin antibody is administered monthly for a period of at least 18 consecutive months. In some embodiments, the anti-sclerostin antibody is administered monthly for a period of at least 24 consecutive months. In some embodiments, the anti-sclerostin antibody is administered monthly for a period of at least 30 consecutive months. In some embodiments, the anti-sclerostin antibody is administered monthly for a period of at least 36 consecutive months.

In some embodiments, the anti-sclerostin antibody is administered monthly for a period of up to 18 years, meaning that the period during which the anti-sclerostin antibody is administered each month does not last more than 18 years. Thus, in one embodiment, the anti-sclerostin antibody is administered monthly for a period of at least 13 consecutive months up to 18 years. In a related embodiment, the anti-sclerostin antibody is administered monthly for a period of at least 18 consecutive months up to 18 years. In a related embodiment, the anti-sclerostin antibody is administered monthly for a period of at least 24 consecutive months up to 18 years. In a related embodiment, the anti-sclerostin antibody is administered monthly for a period of at least 30 consecutive months up to 18 years. In a related embodiment, the anti-sclerostin antibody is administered monthly for a period of at least 36 consecutive months up to 18 years.

An exemplary treatment regimen entails administration of multiple doses, which may be of the same dosage, ranging from about 150-3000 mg, under a dosing schedule of once per month, for a period of at least 13 consecutive months up to 18 years. In some embodiments, the treatment entails monthly administration for at least 18 consecutive months. In some embodiments, the treatment entails monthly administration for at least 24 consecutive months. In some embodiments, the treatment entails monthly administration for at least 30 consecutive months. In some embodiments, the treatment entails monthly administration for at least 36 consecutive months.

In a certain embodiment, the anti-sclerostin antibody is administered monthly at a dose of 150-2500 mg for a period of at least 30 consecutive months up to 18 years

Another exemplary treatment regimen entails administration of multiple doses, which may be of the same dosage, ranging from about 150-3000 mg, under a dosing schedule of once per month, for at least 13 consecutive months and until a treatment target is achieved or reached in the patient. The treatment target is achieved or reached after a certain number of doses are administered. The treatment target may be a complete normalization of bone mineral density, a partial normalization of bone mineral density, or a reduced frequency of bone fracture incidence. Thus, in one embodiment, the monthly administration of the anti-sclerostin antibody increases bone mineral density (BMD) of lumbar spine by 5% or more after 12 consecutive months of therapy.

In another embodiment, the invention provides an anti-sclerostin antibody for use in the treatment of OI, wherein the anti-sclerostin antibody reduces the fracture rate in a patient/patient population compared to a control patient/patient population. Preferably, the anti-sclerostin antibody reduces the fracture rate by at least 10, 20, 30, 35, 40, 50, 60, 70, 80, or 90 percent. In one embodiment the anti-sclerostin antibody reduces the fracture rate by at least 30 percent. In one embodiment, fractures are defined as peripheral or vertebral fractures (including all major, minor, and vertebral clinical fractures; fractures only detected by means of investigations without clinical symptoms are not included), confirmed by radiologic investigation(s). In one embodiment the fracture rate pertains to a population of patients. The patient population and control patient populations are preferably of a size that allow a statistically significant comparison to be made.

The anti-sclerostin antibody is administered intravenously. In a certain embodiment, administration occurs intravenously by way of an infusion.

Measurements of the targets are known in the art. For example, bone mineral density (BMD) may be measured by dual-energy x-ray absorptiometry (DXA), single-energy x-ray absorptiometry (SXA), quantitative computed tomography (CT), ultrasound, and high-resolution peripheral quantitative computed tomography (HR-pQCT). DXA is an x- ray technique that has become the standard for measuring bone density in the art. Though it can be used for measurements of any skeletal site, clinical determinations are usually made of the lumbar spine and hip. Portable DXA machines have been developed that measure the heel (calcaneus), forearm (radius and ulna), or finger (phalanges), and DXA can also be used to measure body composition. Consequently, it has become standard practice to relate the results to “normal” values using T-scores, which compare individual results to those in a young population that is matched for race and gender. Alternatively, Z-scores compare individual results to those of an age-matched population that is also matched for race and gender. Thus, for example, a 60- year-old woman with a Z-score of −1 (1 SD below mean for age) could have a T-score of −2.5 (2.5 SD below mean for a young control group).

Radial bone strength may be measured by micro-finite element analysis (microFEA).

In some embodiments, actual dosage levels of the anti-sclerostin antibody may be varied so as to obtain an amount of the anti-sclerostin antibody which is effective to achieve the desired therapeutic response for a particular patient, composition, and mode of administration, without being toxic to the patient. The selected dosage level will depend upon a variety of pharmacokinetic factors including the activity of the particular compositions employed, the route of administration, the time of administration, the rate of excretion of the particular anti-sclerostin antibody being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective amount” of the anti-sclerostin antibody may result in a decrease in severity of disease symptoms, an increase in frequency and duration of disease symptom-free periods, or a prevention of impairment or disability due to the disease affliction. Accordingly, in one embodiment, the monthly administration of the anti-sclerostin antibody increases bone mineral density (BMD) of lumbar spine by 5% or more after 12 months of therapy.

### Certain Dosing Regimens

In other aspects, the invention provides a method for treating osteogenesis imperfecta (OI) in a human patient comprising administering a therapeutically effective amount of the anti-sclerostin antibody to a human patient in need thereof, wherein the method entails administering a first dosage during an initial dosing period then administering a maintenance dosage thereafter. In some embodiments, the anti-sclerostin antibody is administered monthly for an initial dosing period of 12 months or greater followed by regular dosing at a maintenance dosage.

In some embodiments, the maintenance dosage follows the initial dosing period directly without a dosing holiday between the initial dosing period and the maintenance dosing period. In some embodiments, the maintenance dosage comprises a different dosing regimen from that administered during the initial dosing period; i.e., the maintenance dosage comprises administering the anti-sclerostin antibody at a frequency and/or at a dosage amount that is different from that administered during the initial dosing period.

In some embodiments, the anti-sclerostin antibody is administered during the initial dosing period at a dosage of 20-50 mg/kg, 20-40 mg/kg, or 20 mg/kg. In a particular embodiment, the anti-sclerostin antibody is administered during the initial dosing period at a dosage of 20 mg/kg, administered monthly.

A maintenance dosage may be a dosage of anti-sclerostin antibody administered monthly, about every 2 months, about every 3 months, about every 4 months, about every 6 months, or about every 12 months. A maintenance dosage may be the same amount administered during the initial dosing period (e.g., about 20 mg/kg), or it may be a reduced amount relative to the amount administered during the initial dosing period (e.g., less than 20 mg/kg).

In some embodiments, the maintenance dosage is administered regularly for a period of at least 2, at least 4, at least 6, at least 8, at least 10, or at least 12 consecutive months following the initial dosing period. In some embodiments, the maintenance dosage is administered for a period of at least 18 consecutive months following the initial dosing period. In some embodiments, the maintenance dosage is administered for a period of at least 24 consecutive months following the initial dosing period. In some embodiments, the maintenance dosage is administered for a period of at least 30 consecutive months following the initial dosing period. In some embodiments, the maintenance dosage is administered for a period of at least 36 consecutive months following the initial dosing period. In some embodiments, the maintenance dosage is administered for a period of up to 18 years following the initial dosing period.

Thus, in one embodiment, the anti-sclerostin antibody is administered monthly for a period of 12 months or more then administered bi-monthly thereafter, e.g., for up to 18 years. In a particular embodiment, the anti-sclerostin antibody is administered during the initial dosing period at a dosage of 20 mg/kg, administered monthly, followed by a maintenance dose of 20 mg/kg administered bi-monthly, every three months, every four months, every six months, or every twelve months. In a particular embodiment, the anti-sclerostin antibody is administered during the initial dosing period at a dosage of 20 mg/kg, administered monthly, followed by a maintenance dose of 20 mg/kg administered bi-monthly. In another embodiment, the anti-sclerostin antibody is administered monthly for a period of 12 months or more at a dosage of about 20 mg/kg, then administered monthly thereafter, e.g., for up to 18 years, at a dosage amount of less than 20 mg/kg. In a particular embodiment, the anti-sclerostin antibody is administered during the initial dosing period at a dosage of 20 mg/kg, administered monthly, followed by a maintenance dose of less than 20 mg/kg administered monthly. The maintenance dose of less than 20 mg/kg of the anti-sclerostin antibody may be 2-20 mg/kg, 5-20 mg/kg, 8-20 mg/kg, 10-20 mg/kg, 15-20 mg/kg, 2-19 mg/kg, 5-19 mg/kg, 8-19 mg/kg, 10-19 mg/kg, 15-19 mg/kg, 2-15 mg/kg, 5-15 mg/kg, 8-15 mg/kg, 10-15 mg/kg, 2-10 mg/kg, 5-10 mg/kg, 8-10 mg/kg, less than or about 19 mg/kg, less than or about 15 mg/kg, less than or about 10 mg/kg, less than or about 8 mg/kg, less than or about 2 mg/kg, about 2 mg/kg, about 8 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg of the anti-sclerostin antibody.

A maintenance dosage may be a dosage amount sufficient to maintain BMD and/or stable bone turnover biomarker levels in a patient following the initial dosing period. In some embodiments, a maintenance dosage is a dosage amount sufficient to maintain gains (or the rate of gain) of BMD in a patient following the initial dosing period. A maintenance dosage may be less than the dosage administered to a patient in the initial dosing period. For example, a maintenance dosage may be less than about 50 mg/kg of the anti-sclerostin antibody, less than about 40 mg/kg of the anti-sclerostin antibody, less than about 30 mg/kg of the anti-sclerostin antibody, less than about 20 mg/kg of the anti-sclerostin antibody, less than about 10 mg/kg of the anti-sclerostin antibody, less than about 8 mg/kg of the anti-sclerostin antibody, or less than about 2 mg/kg of the anti-sclerostin antibody. In some embodiments, the maintenance dosage is about 2 mg/kg of the anti-sclerostin antibody, about 8 mg/kg of the anti-sclerostin antibody, about 10 mg/kg of the anti-sclerostin antibody, about 11 mg/kg of the anti-sclerostin antibody, about 12 mg/kg of the anti-sclerostin antibody, about 13 mg/kg of the anti-sclerostin antibody, about 14 mg/kg of the anti-sclerostin antibody, about 15 mg/kg of the anti-sclerostin antibody, about 16 mg/kg of the anti-sclerostin antibody, about 17 mg/kg of the anti-sclerostin antibody, about 18 mg/kg of the anti-sclerostin antibody, about 19 mg/kg of the anti-sclerostin antibody, or about 20 mg/kg of the anti-sclerostin antibody.

As noted above, it is to be understood that ranges such as those provided herein for dosage amounts of an anti-sclerostin antibody comprise endpoints and all subranges therein, including each integer in and between a disclosed range. Thus, the range “from 20 to 50” includes all possible ranges therein (e.g., 21-49, 22-48, 23-47, etc.) as well as each individual integer from 20 to 50 (e.g., 20, 21, 22, 23, 24, etc.). Accordingly, dosage amounts of an anti-sclerostin antibody of 20-50 mg/kg include dosage amounts of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50 mg/kg of the anti-sclerostin antibody. Dosage amounts of an anti-sclerostin antibody of less than 20 mg/kg include dosage amounts of 19, 18, 17, 16, 15, 14, 13, 12, 11, 10, 9, 8, 7, 6, 5, 4, 3, 2, and 1 mg/kg of the antisclerostin antibody.

### Anti-Sclerostin Antibodies

The invention relates to the treatment of bone-related diseases using an anti-sclerostin antibody. In some embodiments, the anti-sclerostin antibody comprises a VH comprising HCDR1, HCDR2, and HCDR3 of the VH sequence of SEQ ID NO:70, and a VL comprising LCDR1, LCDR2, and LCDR3 of the VL sequence of SEQ ID LNO:81.

In one embodiment, the anti-sclerostin antibody comprises: (a) an HCDR1 having the amino acid sequence of SEQ ID NO:4 (GFTFRSHWLS); (b) an HCDR2 having the amino acid sequence of SEQ ID NO:15 (WVSNINYDGSSTYYADSVKG); (c) an HCDR3 having the amino acid sequence of SEQ ID NO:26 (DTYLHFDY); (d) an LCDR1 having the amino acid sequence of SEQ ID NO:37 (TGTSSDVGDINDVS); (e) an LCDR2 having the amino acid sequence of SEQ ID NO:48 (LMIYDVNNRPS); and (f) an LCDR3 having the amino acid sequence of SEQ ID NO:59 (QSYAGSYLSE).

The CDRs within a VH or VL sequence can be delineated by different classification and numbering systems. CDRs may therefore be referred to by IMGT, Kabat, Chothia, AbM, Contact, a combination of these systems, or another system. See, for example: Kabat (Kabat, E. A., et al., 1991 Sequences of Proteins of Immunological Interest, Fifth Edition, U.S. Department of Health and Human Services, NIH Publication No. 91-3242); Chothia (Chothia et al. (1987) J Mol Biol 196: 901-17); IMGT (Leffanc et al. (2003) Dev Comp Immunol 27: 55-77); AbM (Martin and Thornton J Bmol Biol 263: 800-15, 1996); and Contact (MacCallum, R. M., Martin, A. C. R. and Thornton, J. T. “Antibody-antigen interactions: Contact analysis and binding site topography” J. Mol. Biol. 262:732-745). The overlap and differences between these classification systems is described (see e.g. Lefranc et al. (2003) Dev Comp Immunol 27: 55-77; Honegger and Pluckthun, J Mol Biol (2001) 309:657-70; International ImMunoGeneTics (IMGT) database; web resources, www.imgt.org). One skilled in the art can identify the CDRs in a VH and VL sequence using methods in the art. For example, CDRs can be delineated using available programs such as abYsis (www.abysis.org; Swindells et al. J Mol Biol. 2017 Feb. 3; 429(3):356-64).

Table 1 shows the amino acid sequences of the CDR sequences of setrusumab (an antibody having the VH of SEQ ID NO: 70 and the VL of SEQ ID NO: 81) as defined according to the Kabat, Chothia, IMGT, AbM, and Contact systems.

TABLE 1
Example CDR sequences delineated by different classification systems

| Feature | Kabat | Chothia | IMGT | AbM | Contact |
|---|---|---|---|---|---|
| HCDR1 | SHWLS | GFTFRSH | GFTFRSHW | GFTFRSHWLS | RSHWLS |
| | (SEQ ID NO: | (SEQ ID NO: 182) | (SEQ ID NO: 184) | (SEQ ID NO: 4) | (SEQ ID NO: 190) |
| | 178) | | | | |
| HCDR2 | NINYDGSSTY | NYDGSS | INYDGSST | NINYDGSSTY | WVSNINYDGSST |
| | YADSVKG | (SEQ ID NO: 183) | (SEQ ID NO: 185) | (SEQ ID NO: 189) | Y |
| | (SEQ ID NO: | | | | (SEQ ID NO: 191) |
| | 179) | | | | |
| HCDR3 | DTYLHFDY | DTYLHFDY | ARDTYLHFDY | DTYLHFDY | ARDTYLHFD |
| | (SEQ ID NO: 26) | (SEQ ID NO: 26) | (SEQ ID NO: 186) | (SEQ ID NO: 26) | (SEQ ID NO: 192) |
| LCDR1 | TGTSSDVGDI | TGTSSDVGDIND | SSDVGDIND | VS | VGDINDVSWY |
| | NDVS | VS (SEQ ID NO: | (SEQ ID NO: 187) | TGTSSDVGDIND | (SEQ ID NO: 193) |
| | (SEQ ID NO: | 37) | | (SEQ ID NO: 37) | |
| | 37)) | | | | |
| LCDR2 | DVNNRPS | DVNNRPS | DV | DVNNRPS | LMIYDVNNRP |
| | (SEQ ID NO: | (SEQ ID NO: 180) | (SEQ ID NO: 188) | (SEQ ID NO: 180) | (SEQ ID NO: 194) |
| | 180) | | | | |
| LCDR3 | QSYAGSYLSE | QSYAGSYLSEV | (SEQ ID NO: | QSYAGSYLSEV | QSYAGSYLSE |
| | V | (SEQ ID NO: 181) | 181) | (SEQ ID NO: 181) | (SEQ ID NO: 59) |
| | (SEQ ID NO: | | QSYAGSYLSEV | | |
| | 181) | | | | |

In one embodiment, the setrusumab CDRs are delineated by Kabat. Accordingly, in one embodiment, the anti-sclerostin antibody comprises: (a) an HCDR1 having the amino acid sequence of SEQ ID NO:178; (b) an HCDR2 having the amino acid sequence of SEQ ID NO:179; (c) an HCDR3 having the amino acid sequence of SEQ ID NO:26; (d) an LCDR1 having the amino acid sequence of SEQ ID NO:37; (e) an LCDR2 having the amino acid sequence of SEQ ID NO:180; and (f) an LCDR3 having the amino acid sequence of SEQ ID NO:181.

In another embodiment, the setrusumab CDRs are delineated by Chothia. Accordingly, in one embodiment, the anti-sclerostin antibody comprises: (a) an HCDR1 having the amino acid sequence of SEQ ID NO:182; (b) an HCDR2 having the amino acid sequence of SEQ ID NO:183; (c) an HCDR3 having the amino acid sequence of SEQ ID NO:26; (d) an LCDR1 having the amino acid sequence of SEQ ID NO:37; (e) an LCDR2 having the amino acid sequence of SEQ ID NO:180; and (f) an LCDR3 having the amino acid sequence of SEQ ID NO:181.

In another embodiment, the setrusumab CDRs of the anti-sclerostin antibody are defined to encompass CDRs as defined by both Kabat and Chothia. Each CDR sequence therefore spans the sequence from the most N-terminal residue out of the Kabat and Chothia-defined CDRs to the most C-terminal residue out of the Kabat and Chothia-defined CDRs. Accordingly, in one embodiment, the anti-sclerostin antibody comprises: (a) an HCDR1 having the amino acid sequence of SEQ ID NO:4; (b) an HCDR2 having the amino acid sequence of SEQ ID NO:179; (c) an HCDR3 having the amino acid sequence of SEQ ID NO:26; (d) an LCDR1 having the amino acid sequence of SEQ ID NO:37; (e) an LCDR2 having the amino acid sequence of SEQ ID NO:180; and (f) an LCDR3 having the amino acid sequence of SEQ ID NO:181.

In one embodiment, the setrusumab CDRs are delineated by IMGT. Accordingly, in one embodiment, the anti-sclerostin antibody comprises: (a) an HCDR1 having the amino acid sequence of SEQ ID NO:184; (b) an HCDR2 having the amino acid sequence of SEQ ID NO:185; (c) an HCDR3 having the amino acid sequence of SEQ ID NO:186; (d) an LCDR1 having the amino acid sequence of SEQ ID NO: 187; (e) an LCDR2 having the amino acid sequence of SEQ ID NO:188; and (f) an LCDR3 having the amino acid sequence of SEQ ID NO:181.

In one embodiment, the setrusumab CDRs are delineated by AbM. Accordingly, in one embodiment, the anti-sclerostin antibody comprises: (a) an HCDR1 having the amino acid sequence of SEQ ID NO:4; (b) an HCDR2 having the amino acid sequence of SEQ ID NO:189; (c) an HCDR3 having the amino acid sequence of SEQ ID NO:26; (d) an LCDR1 having the amino acid sequence of SEQ ID NO:37; (e) an LCDR2 having the amino acid sequence of SEQ ID NO:180; and (f) an LCDR3 having the amino acid sequence of SEQ ID NO:181.

In one embodiment, the setrusumab CDRs are delineated by Contact. Accordingly, in one embodiment, the anti-sclerostin antibody comprises: (a) an HCDR1 having the amino acid sequence of SEQ ID NO:190; (b) an HCDR2 having the amino acid sequence of SEQ ID NO:191; (c) an HCDR3 having the amino acid sequence of SEQ ID NO:192; (d) an LCDR1 having the amino acid sequence of SEQ ID NO: 193; (e) an LCDR2 having the amino acid sequence of SEQ ID NO:194; and (f) an LCDR3 having the amino acid sequence of SEQ ID NO:59.

In a certain embodiment, the anti-sclerostin antibody comprises: a VH polypeptide amino acid sequence having at least 95 percent identity to the amino acid sequence of SEQ ID NO: 70.

In a certain embodiment, the anti-sclerostin antibody comprises: a VL polypeptide amino acid sequence having at least 95 percent identity to the amino acid sequence of SEQ ID NO: 81.

In a certain embodiment, the anti-sclerostin antibody comprises: a VH polypeptide amino acid sequence having at least 95 percent identity to the amino acid sequence of SEQ ID NO: 70 and a VL polypeptide amino acid sequence having at least 95 percent identity to the amino acid sequence of SEQ ID NO: 81.

In a certain embodiment, the anti-sclerostin antibody comprises: the VH polypeptide amino acid sequence of SEQ ID NO: 70 and the VL polypeptide amino acid sequence of SEQ ID NO: 81.

In a certain embodiment, the anti-sclerostin antibody comprises: the heavy chain polypeptide amino acid sequence of SEQ ID NO: 114 (MAWVWTLPFLMAAAQSVQAQVQLVESGGGLVQPGGSLRLSCAASGFTFRSHWLS WVRQAPGKGLEWVSNINYDGSSTYYADSVKGRFTISRDNSKNTLYLQMNSLRAEDT AVYYCARDTYLHFDYWGQGTLVTVSSASTKGPSVFPLAPCSRSTSESTAALGCLVK DYFPEPVTVSWNSGALTSGVHTFPAVLQSSGLYSLSSVVTVPSSNFGTQTYTCNVDH KPSNTKVDKTVERKCCVECPPCPAPPVAGPSVFLFPPKPKDTLMISRTPEVTCVVVD VSHEDPEVQFNWYVDGVEVHNAKTKPREEQFNSTFRVVSVLTVVHQDWLNGKEYK CKVSNKGLPAPIEKTISKTKGQPREPQVYTLPPSREEMTKNQVSLTCLVKGFYPSDIA VEWESNGQPENNYKTTPPMLDSDGSFFLYSKLTVDKSRWQQGNVFSCSVMHEALH NHYTQKSLSLSPGK) or SEQ ID NO: 172 and the light chain polypeptide amino acid sequence of SEQ ID NO: 125 (MSVLTQVLALLLLWLTGTRCDIALTQPASVSGSPGQSITISCTGTSSDVGDINDVSW YQQHPGKAPKLMIYDVNNRPSGVSNRFSGSKSGNTASLTISGLQAEDEADYYCQSY AGSYLSEVFGGGTKLTVLGQPKAAPSVTLFPPSSEELQANKATLVCLISDFYPGAVTV AWKADSSPVKAGVETTTPSKQSNNKYAASSYLSLTPEQWKSHRSYSCQVTHEGSTV EKTVAPTECS) or SEQ ID NO: 173.

In one embodiment, the anti-sclerostin antibody of the invention is a monoclonal anti-sclerostin antibody. In one embodiment the anti-sclerostin antibody of the invention is a human or humanized monoclonal anti-sclerostin antibody. Alternatively, the antibody can be, for example, a chimeric antibody.

In a preferred embodiment, the anti-sclerostin antibody is the antibody setrusumab, which is a human anti-sclerostin monoclonal antibody. The VH and VL sequences of setrusumab comprise: the VH polypeptide amino acid sequence of SEQ ID NO: 70 and the VL polypeptide amino acid sequence of SEQ ID NO: 81. The heavy and light chain sequences of setrusumab comprise: the heavy chain polypeptide amino acid sequence of SEQ ID NO: 172 and the light chain polypeptide amino acid sequence of SEQ ID NO: 173.

Sequence of setrusumab VH
(SEQ ID NO: 70):
QVQLVESGGGLVQPGGSLRLSCAASGFTFRSHWLSWVRQAPGKGL
EWVSNINYDGSSTYYADSVKGRFTISRDNSKNTLYLQMNSLRAED
TAVYYCARDTYLHFDYWGQGTLVTVSS
Sequence of setrusumab VL
(SEQ ID NO: 81):
DIALTQPASVSGSPGQSITISCTGTSSDVGDINDVSWYQQHPGKA
PKLMIYDVNNRPSGVSNRFSGSKSGNTASLTISGLQAEDEADYYC
QSYAGSYLSEVFGGGTKLTVL
Sequence of setrusumab H-chain
(SEQ ID NO: 172):
QVQLVESGGGLVQPGGSLRLSCAASGFTFRSHWLSWVRQAPGKGL
EWVSNINYDGSSTYYADSVKGRFTISRDNSKNTLYLQMNSLRAED
TAVYYCARDTYLHFDYWGQGTLVTVSSASTKGPSVFPLAPCSRST
SESTAALGCLVKDYFPEPVTVSWNSGALTSGVHTFPAVLQSSGLY
SLSSVVTVPSSNFGTQTYTCNVDHKPSNTKVDKTVERKCCVECPP
CPAPPVAGPSVFLFPPKPKDTLMISRTPEVTCVVVDVSHEDPEVQ
FNWYVDGVEVHNAKTKPREEQFNSTFRVVSVLTVVHQDWLNGKEY
KCKVSNKGLPAPIEKTISKTKGQPREPQVYTLPPSREEMTKNQVS
LTCLVKGFYPSDIAVEWESNGQPENNYKTTPPMLDSDGSFFLYSK
LTVDKSRWQQGNVFSCSVMHEALHNHYTQKSLSLSPGK
Sequence of setrusumab L-chain
(SEQ ID NO: 173):
DIALTQPASVSGSPGQSITISCTGTSSDVGDINDVSWYQQHPGKA
PKLMIYDVNNRPSGVSNRFSGSKSGNTASLTISGLQAEDEADYYC
QSYAGSYLSEVFGGGTKLTVLGQPKAAPSVTLFPPSSEELQANKA
TLVCLISDFYPGAVTVAWKADSSPVKAGVETTTPSKQSNNKYAAS
SYLSLTPEQWKSHRSYSCQVTHEGSTVEKTVAPTECS
In a related embodiment, the anti-sclerostin
antibody comprises: the VH polypeptide amino
acid sequence of SEQ ID NO: 70 and the VL
polypeptide amino acid sequence of
SEQ ID NO: 195
(DIALTQPASVSGSPGQSITISCTGTSSDVGDINDVSWYQQHPGK
APKLMIYDVNNRPSGVSNRFSGSKSGNTASLTISGLQAEDEADYY
CQSYAGSYLSEVFGGGTKLTVLGQ).

The VL polypeptide amino acid sequence of SEQ ID NO:81 is delineated according to the Kabat system. The VL polypeptide amino acid sequence of 195 is an alternative delineation of the VL domain, which encompasses the sequence according to Kabat, but also includes an additional two amino acids.

Additional characteristics of the anti-sclerostin antibodies used in the present invention, such as setrusumab, are described in WO2009/047356, which disclosure, discussion and data are hereby incorporated by reference thereto. By way of example only, the antibodies may exhibit at least one of the following functional properties: the antibody blocks the inhibitory effect of sclerostin in a cell based wnt signaling assay, the antibody blocks the inhibitory effect of sclerostin in a cell based mineralization assay, the antibody blocks the inhibitory effect of sclerostin in Smad1 phosphorylation assay, the antibody inhibits binding of sclerostin to the LRP-6, and the antibody increases bone formation and mass and density. As noted above, these properties are described in detail in WO2009/047356.

In some embodiments, the anti-sclerostin antibody comprises (a) a VH polypeptide sequence having at least 90 percent sequence identity to the amino acid sequence set forth in SEQ ID NO: 198; and/or (b) a VL polypeptide sequence having at least 90 percent sequence identity to the amino acid sequence set forth in SEQ ID NO: 199. In one embodiment, the anti-sclerostin antibody comprises a VH polypeptide sequence comprising the amino acid sequence set forth in SEQ ID NO: 198 and a VL polypeptide sequence comprising the amino acid sequence set forth in SEQ ID NO: 199. In some embodiments, the antisclerostin antibody comprises a heavy chain (HC) polypeptide sequence having at least 90 percent sequence identity to the amino acid sequence set forth in SEQ ID NO: 196 and/or a light chain (LC) polypeptide sequence having at least 90 percent sequence identity to the amino acid sequence set forth in SEQ ID NO: 197. In one embodiment, the anti-sclerostin antibody comprises a HC polypeptide sequence comprising the amino acid sequence set forth in SEQ ID NO: 196 and a LC polypeptide sequence comprising the amino acid sequence set forth in SEQ ID NO: 197.

In some embodiments, the anti-sclerostin antibody comprises (a) a VH polypeptide sequence having at least 90 percent sequence identity to the amino acid sequence set forth in SEQ ID NO: 202; and/or (b) a VL polypeptide sequence having at least 90 percent sequence identity to the amino acid sequence set forth in SEQ ID NO: 203. In one embodiment, the anti-sclerostin antibody comprises a VH polypeptide sequence comprising the amino acid sequence set forth in SEQ ID NO: 202 and a VL polypeptide sequence comprising the amino acid sequence set forth in SEQ ID NO: 203. In some embodiments, the antisclerostin antibody comprises a heavy chain (HC) polypeptide sequence having at least 90 percent sequence identity to the amino acid sequence set forth in SEQ ID NO: 200 and/or a light chain (LC) polypeptide sequence having at least 90 percent sequence identity to the amino acid sequence set forth in SEQ ID NO: 201. In one embodiment, the anti-sclerostin antibody comprises a HC polypeptide sequence comprising the amino acid sequence set forth in SEQ ID NO: 200 and a LC polypeptide sequence comprising the amino acid sequence set forth in SEQ ID NO: 201.

Various anti-sclerostin antibody polypeptide sequences are set forth below:

HC polypeptide sequence:
(SEQ ID NO: 196)
EVQLVQSGAEVKKPGASVKVSCKASGYTFTDYNMHWVRQAPGQGL
EWMGEINPNSGGAGYNQKFKGRVTMTTDTSTSTAYMELRSLRSDD
TAVYYCARLGYDDIYDDWYFDVWGQGTTVTVSSASTKGPSVFPLA
PCSRSTSESTAALGCLVKDYFPEPVTVSWNSGALTSGVHTFPAVL
QSSGLYSLSSVVTVPSSNFGTQTYTCNVDHKPSNTKVDKTVERKC
CVECPPCPAPPVAGPSVFLFPPKPKDTLMISRTPEVTCVVVDVSH
EDPEVQFNWYVDGVEVHNAKTKPREEQFNSTFRVVSVLTVVHQDW
LNGKEYKCKVSNKGLPAPIEKTISKTKGQPREPQVYTLPPSREEM
TKNQVSLTCLVKGFYPSDIAVEWESNGQPENNYKTTPPMLDSDGS
FFLYSKLTVDKSRWQQGNVFSCSVMHEALHNHYTQKSLSLSPGK
LC polypeptide sequence:
(SEQ ID NO: 197)
DIQMTQSPSSLSASVGDRVTITCRASQDISNYLNWYQQKPGKAPK
LLIYYTSRLLSGVPSRFSGSGSGTDFTLTISSLQPEDFATYYCQQ
GDTLPYTFGGGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
VH polypeptide sequence:
(SEQ ID NO: 198)
EVQLVQSGAEVKKPGASVKVSCKASGYTFTDYNMHWVRQAPGQGL
EWMGEINPNSGGAGYNQKFKGRVTMTTDTSTSTAYMELRSLRSDD
TAVYYCARLGYDDIYDDWYFDVWGQGTTVTVSS
VL
polypeptide sequence:
(SEQ ID NO: 199)
DIQMTQSPSSLSASVGDRVTITCRASQDISNYLNWYQQKPGKAPK
LLIYYTSRLLSGVPSRFSGSGSGTDFTLTISSLQPEDFATYYCQQ
GDTLPYTFGGGTKVEIKRTV
HC polypeptide sequence:
(SEQ ID NO: 200)
QVQLVQSGAEVKKPGASVKVSCKVSGFPIKDTFQHWVRQAPGKGL
EWMGWSDPEIGDTEYASKFQGRVTMTEDTSTDTAYMELSSLRSED
TAVYYCATGDTTYKFDFWGQGTTVTVSSASTKGPSVFPLAPCSRS
TSESTAALGCLVKDYFPEPVTVSWNSGALTSGVHTFPAVLQSSGL
YSLSSVVTVPSSSLGTKTYTCNVDHKPSNTKVDKRVESKYGPPCP
PCPAPEFLGGPSVFLFPPKPKDTLMISRTPEVTCVVVDVSQEDPE
VQFNWYVDGVEVHNAKTKPREEQFNSTYRVVSVLTVLHQDWLNGK
EYKCKVSNKGLPSSIEKTISKAKGQPREPQVYTLPPSQEEMTKNQ
VSLTCLVKGFYPSDIAVEWESNGQPENNYKTTPPVLDSDGSFFLY
SRLTVDKSRWQEGNVFSCSVMHEALHNHYTQKSLSLSLG
LC polypeptide sequence:
(SEQ ID NO: 201)
DIQMTQSPSSLSASVGDRVTITCKASQDVHTAVAWYQQKPGKAPK
LLIYWASTRWTGVPSRFSGSGSGTDFTLTISSLQPEDFATYYCQQ
YSDYPWTFGGGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
VH polypeptide sequence:
(SEQ ID NO: 202)
QVQLVQSGAEVKKPGASVKVSCKVSGFPIKDTFQHWVRQAPGKGL
EWMGWSDPEIGDTEYASKFQGRVTMTEDTSTDTAYMELSSLRSED
TAVYYCATGDTTYKFDFWGQGTTVTVSS
VL polypeptide sequence:
(SEQ ID NO: 203)
DIQMTQSPSSLSASVGDRVTITCKASQDVHTAVAWYQQKPGKAPK
LLIYWASTRWTGVPSRFSGSGSGTDFTLTISSLQPEDFATYYCQQ
YSDYPWTFGGGTKVEIKRTV

In some embodiments, the anti-sclerostin antibody comprises (a) a heavy chain variable region CDR1 comprising an amino acid sequence set forth in SEQ ID NO: 4; (b) a heavy chain variable region CDR2 comprising an amino acid sequence set forth in SEQ ID NO: 15; (c) a heavy chain variable region CDR3 comprising an amino acid sequence set forth in SEQ ID NO: 26; (d) a light chain variable region CDR1 comprising an amino acid sequence set forth in SEQ ID NO: 37; (e) a light chain variable region CDR2 comprising an amino acid sequence set forth in SEQ ID NO: 48; and (f) a light chain variable region CDR3 comprising an amino acid sequence set forth in SEQ ID NO: 59.

In some embodiments, the anti-sclerostin antibody comprises (a) a heavy chain variable region CDR1 comprising an amino acid sequence set forth in SEQ ID NO: 178; (b) a heavy chain variable region CDR2 comprising an amino acid sequence set forth in SEQ ID NO: 179; (c) a heavy chain variable region CDR3 comprising an amino acid sequence set forth in SEQ ID NO: 26; (d) a light chain variable region CDR1 comprising an amino acid sequence set forth in SEQ ID NO: 37; (e) a light chain variable region CDR2 comprising an amino acid sequence set forth in SEQ ID NO: 180; and (f) a light chain variable region CDR3 comprising an amino acid sequence set forth in SEQ ID NO: 181.

In some embodiments, the anti-sclerostin antibody comprises (a) a heavy chain variable region CDR1 comprising an amino acid sequence set forth in SEQ ID NO: 4; (b) a heavy chain variable region CDR2 comprising an amino acid sequence set forth in SEQ ID NO: 179; (c) a heavy chain variable region CDR3 comprising an amino acid sequence set forth in SEQ ID NO: 26; (d) a light chain variable region CDR1 comprising an amino acid sequence set forth in SEQ ID NO: 37; (e) a light chain variable region CDR2 comprising an amino acid sequence set forth in SEQ ID NO: 180; and (f) a light chain variable region CDR3 comprising an amino acid sequence set forth in SEQ ID NO: 181.

In some embodiments, the anti-sclerostin antibody is one described in U.S. Pat. Nos. 7,879,322, 8,246,953, 8,486,661, 8,003,108, 7,592,429, 8,017,120, or 10,449,250; International Patent Application Number WO2018/115879A1, WO2018/115880A1, WO2013/019954A1, WO2008/115732A2, or WO2015/087187A1; or U.S. Patent Application Publication No. 20110044978A1; which are hereby incorporated in their entirety by reference thereto.

In some embodiments, the anti-sclerostin antibody is selected from setrusumab, romosozumab, and blosozumab.

In relation to an antibody that “blocks the inhibitory effect of sclerostin in a cell based wnt signaling assay”, this is intended to refer to an antibody that restores wnt induced signaling in the presence of sclerostin in a cell-based super top flash (STF) assay with an IC50 less than 1 mM, 100 nM, 20 nM, 10 nM or less. WO2009/047356 describes said wnt STF assay.

In relation to an antibody that “blocks the inhibitory effect of sclerostin in a cell based mineralization assay”, this is intended to refer to an antibody that restores BMP2 induced mineralization in the presence of sclerostin in a cell-based assay with an IC50 less than 1 mM, 500 nM, 100 nM, 10 nM, 1 nM or less.

In relation to an antibody that “blocks the inhibitory effect of sclerostin in Smad1 phosphorylation assay”, this is intended to refer to an antibody that restores BMP6 induced Smad1 phosphorylation in the presence of sclerostin in a cell based assay with an IC50 less than 1 mM, 500 nM, 100 nM, 10 nM, 1 nM or less.

In relation to an antibody that “inhibits binding of sclerostin to the LRP-6”, this is intended to refer to an antibody that inhibits sclerostin binding to LRP-6 with a IC50 of 1 mM, 500 nM, 100 nM, 10 nM, 5 nM, 3 nM, 1 nM or less.

In relation to an antibody that “increases bone formation and mass and density”, this is intended to refer to an antibody that is capable of reaching bone formation, mass and density at the level of daily intermittent treatment with high anabolic dose of PTH, such as a daily intermittent treatment with 100 μg/kg of hPTH.

In one embodiment, the anti-sclerostin antibody of the invention increases bone formation and/or reduces bone resorption.

In one aspect, the present invention provides a method for treating a bone-related disease in a human patient comprising administering to the human patient a therapeutically effective amount of an anti-sclerostin antibody each month for a period of at least 13 consecutive months, and wherein the antibody cross-blocks an anti-sclerostin antibody that comprises a VH comprising an HCDR1, an HCDR2, and an HCDR3 domain of the VH sequence of SEQ ID NO:70, and a VL comprising an LCDR1, an LCDR2, and an LCDR3 domain of the VL sequence of SEQ ID NO:81.

In another aspect, the invention provides an anti-sclerostin antibody for use in the treatment of a bone-related disease, wherein a therapeutically effective amount of the anti-sclerostin antibody is administered each month for a period of at least 13 consecutive months, and wherein the antibody cross-blocks an anti-sclerostin antibody that comprises a VH comprising an HCDR1, an HCDR2, and an HCDR3 domain of the VH sequence of SEQ ID NO:70, and a VL comprising an LCDR1, an LCDR2, and an LCDR3 domain of the VL sequence of SEQ ID NO:81.

In another aspect, the invention provides use of an anti-sclerostin antibody for the manufacture of a medicament for the treatment of a bone-related disease, wherein the treatment comprises administering a therapeutically effective amount of the anti-sclerostin antibody each month for a period of at least 13 consecutive months, and wherein the antibody cross-blocks an anti-sclerostin antibody that comprises a VH comprising an HCDR1, an HCDR2, and an HCDR3 domain of the VH sequence of SEQ ID NO:70, and a VL comprising an LCDR1, an LCDR2, and an LCDR3 domain of the VL sequence of SEQ ID NO:81.

In one embodiment, the anti-sclerostin antibody used according to the invention binds sclerostin with an affinity of less than or equal to 10⁻¹¹ M (measured by Biacore) and cross-blocks an anti-sclerostin antibody that comprises a VH comprising an HCDR1, an HCDR2, and an HCDR3 domain of the VH sequence of SEQ ID NO:70, and a VL comprising an LCDR1, an LCDR2, and an LCDR3 domain of the VL sequence of SEQ ID NO:81.

According to the invention, a cross-blocking antibody or other binding agent according to the invention binds to sclerostin in the Biacore cross-blocking assay, described below, such that the recorded binding of the combination (mixture) of the antibodies or binding agents is between 80% and 0.1% (e.g. 80% to 4%) of the maximum theoretical binding, specifically between 75% and 0.1% (e.g. 75% to 4%) of the maximum theoretical binding, and more specifically between 70% and 0.1% (e.g. 70% to 4%), and more specifically between 65% and 0.1% (e.g. 65% to 4%) of maximum theoretical binding (as defined above) of the two antibodies or binding agents in combination.

The following generally describes a suitable Biacore assay for determining whether an antibody or other binding agent cross-blocks or is capable of cross-blocking antibodies according to the invention. It will be appreciated that the assay can be used with any of the sclerostin binding antibodies described herein.

The Biacore machine (for example the Biacore 3000) is operated in line with the manufacturer's recommendations.

Sclerostin may be coupled to e.g. a CM5 Biacore chip by way of routinely used amine coupling chemistry, e.g. EDC-NHS amine coupling, to create a sclerostin-coated surface. In order to obtain measurable levels of binding, typically 200-800 resonance units of sclerostin may be coupled to the chip (this amount gives measurable levels of binding and is at the same time readily saturable by the concentrations of test reagent being used).

An alternative way of attaching sclerostin to the Biacore chip is by using a “tagged” version of sclerostin, for example N-terminal or C-terminal His-tagged Sclerostin. In this format, an anti-His antibody would be coupled to the Biacore chip and then the His-tagged sclerostin would be passed over the surface of the chip and captured by the anti-His antibody.

The two antibodies to be assessed for their ability to cross-block each other are mixed in a stoichiometric amount, e.g. at a one to one molar ratio, of binding sites in a suitable buffer to create the test mixture. The buffer used is typically a buffer which is normally used in protein chemistry, such as e.g. PBS (136 mM NaCl, 2.7 mM KCl, 10 mM Na₂HPO₄, 1.76 mM KH₂PO₄, pH 7.4). When calculating the concentrations on a binding site-basis the molecular weight of an antibody is assumed to be the total molecular weight of the antibody divided by the number of target (i.e. sclerostin) binding sites on that antibody.

The concentration of each antibody in the test mixture should be high enough to ensure saturation of the binding sites for that antibody on the sclerostin molecules which are bound on the Biacore chip. The antibodies in the mixture are at the same molar concentration (on a binding basis) and that concentration would typically be between 1.0 mM and 1.5 mM (on a binding site basis).

Separate solutions containing the separate antibodies on their own are also prepared. The buffer used for these separate solutions should be the same buffer and at the same concentration as was used for the test mixture.

The test mixture is passed over the sclerostin-coated Biacore chip and the binding recorded. The bound antibodies are thereafter removed by treating the chip with e.g. an acid, such as 30 mM HCl for about 1 minute. It is important that the sclerostin molecules which are bound to the chip are not damaged. The solution of the first antibody alone is then passed over the sclerostin-coated surface and the binding is recorded. Thereafter, the chip is treated to remove all of the bound antibody without damaging the chip-bound sclerostin, e.g. by way of above mentioned acid treatment. The solution of the second antibody alone is then passed over the sclerostin-coated surface and the amount of binding recorded.

The maximal theoretical binding can be defined as the sum of the binding to sclerostin of each antibody separately. This is then compared to the actual binding of the mixture of antibodies measured. If the actual binding is lower than that of the theoretical binding, the two antibodies are cross-blocking each other.

An antibody is defined as cross-blocking in the ELISA assay as described below, if the solution phase anti-sclerostin antibody is able to cause a reduction of between 60% and 100%, specifically between 70% and 100%, and more specifically between 80% and 100%, of the sclerostin detection signal (i.e. the amount of sclerostin bound by the coated antibody) as compared to the sclerostin detection signal obtained in the absence of the solution phase anti-sclerostin antibody (i.e. the positive control wells).

Cross-blocking of an anti-sclerostin antibody or another sclerostin binding agent may also be detected by using an ELISA assay. The general principle of the ELISA-assay involves coating an anti-sclerostin antibody onto the wells of an ELISA plate. An excess amount of a second, potentially cross-blocking, anti-sclerostin antibody is then added in solution (i.e. not bound to the ELISA plate). A limited amount of sclerostin is then added to the wells.

The antibody which was coated onto the wells and the antibody in solution will compete for binding of the limited number of sclerostin molecules. The plate is then washed to remove sclerostin that has not bound to the coated antibody and to also remove the second, solution phase, antibody as well as any complexes formed between the second, solution phase antibody and sclerostin. The amount of bound sclerostin is then measured using an appropriate sclerostin detection reagent. An antibody in solution that is able to cross-block the coated antibody will be able to cause a decrease in the number of sclerostin molecules that the coated antibody can bind relative to the number of sclerostin molecules that the coated antibody can bind in the absence of the second, solution phase, antibody.

This assay is described in more detail further below for two antibodies termed Ab-X and Ab-Y. In the instance where Ab-X is chosen to be the immobilized antibody, it is coated onto the wells of the ELISA plate, after which the plates are blocked with a suitable blocking solution to minimize non-specific binding of reagents that are subsequently added. An excess amount of Ab-Y is then added to the ELISA plate such that the moles of Ab-Y sclerostin binding sites per well are at least 10-fold higher than the moles of Ab-X sclerostin binding sites that were used, per well, during the coating of the ELISA plate. Sclerostin is then added such that the moles of sclerostin added per well are at least 25-fold lower than the moles of Ab-X sclerostin binding sites that were used for coating each well. Following a suitable incubation period, the ELISA plate is washed and a sclerostin detection reagent is added to measure the amount of sclerostin specifically bound by the coated anti-sclerostin antibody (in this case Ab-X). The background signal for the assay is defined as the signal obtained in wells with the coated antibody (in this case Ab-X), second solution phase antibody (in this case Ab-Y), sclerostin buffer only (i.e. no sclerostin) and sclerostin detection reagents. The positive control signal for the assay is defined as the signal obtained in wells with the coated antibody (in this case Ab-X), second solution phase antibody buffer only (i.e. no second solution phase antibody), sclerostin and sclerostin detection reagents. The ELISA assay needs to be run in such a manner so as to have the positive control signal be at least 6 times the background signal.

To avoid any artifacts (e.g. significantly different affinities between Ab-X and Ab-Y for sclerostin) resulting from the choice of which antibody to use as the coating antibody and which to use as the second (competitor) antibody, the cross-blocking assay needs to be run in two formats: 1) format 1 is where Ab-X is the antibody that is coated onto the ELISA plate and Ab-Y is the competitor antibody that is in solution and 2) format 2 is where Ab-Y is the antibody that is coated onto the ELISA plate and Ab-X is the competitor antibody that is in solution.

In another aspect, the present invention provides a method for treating a bone-related disease in a human patient comprising administering to the human patient a therapeutically effective amount of an anti-sclerostin antibody each month for a period of at least 13 consecutive months, and wherein the antibody binds to the same epitope as an anti-sclerostin antibody that comprises a VH comprising an HCDR1, an HCDR2, and an HCDR3 domain of the VH sequence of SEQ ID NO:70, and a VL comprising an LCDR1, an LCDR2, and an LCDR3 domain of the VL sequence of SEQ ID NO:81.

In another aspect, the invention provides an anti-sclerostin antibody for use in the treatment of a bone-related disease, wherein a therapeutically effective amount of the anti-sclerostin antibody is administered each month for a period of at least 13 consecutive months, and wherein the antibody binds to the same epitope as an anti-sclerostin antibody that comprises a VH comprising an HCDR1, an HCDR2, and an HCDR3 domain of the VH sequence of SEQ ID NO:70, and a VL comprising an LCDR1, an LCDR2, and an LCDR3 domain of the VL sequence of SEQ ID NO:81.

In another aspect, the invention provides use of an anti-sclerostin antibody for the manufacture of a medicament for the treatment of a bone-related disease, wherein the treatment comprises administering a therapeutically effective amount of the anti-sclerostin antibody each month for a period of at least 13 consecutive months, and wherein the antibody binds to the same epitope as an anti-sclerostin antibody that comprises a VH comprising an HCDR1, an HCDR2, and an HCDR3 domain of the VH sequence of SEQ ID NO:70, and a VL comprising an LCDR1, an LCDR2, and an LCDR3 domain of the VL sequence of SEQ ID NO:81.

In one embodiment, the anti-sclerostin antibody used according to the invention binds sclerostin with an affinity of less than or equal to 10⁻¹¹ M (measured by Biacore) and binds to the same epitope an anti-sclerostin antibody that comprises a VH comprising an HCDR1, an HCDR2, and an HCDR3 domain of the VH sequence of SEQ ID NO:70, and a VL comprising an LCDR1, an LCDR2, and an LCDR3 domain of the VL sequence of SEQ ID NO:81.

Standard assays to determine the epitope of an antibodies are known in the art, including for example, X-ray crystallography, nuclear magnetic resonance (NMR), hydrogen-deuterium exchange coupled to mass spectrometry, peptide-based approaches, or mutagenesis-based approaches (all discussed in Abbott et al. “Current approaches to fine mapping of antigen-antibody interactions.” Immunology vol. 142,4 (2014): 526-35, and references therein).

### Pharmaceutical Compositions

In one embodiment, the anti-sclerostin antibody is provided as a pharmaceutical composition. The pharmaceutical composition may be formulated with a pharmaceutically acceptable carrier. Thus, in one aspect, the invention provides a pharmaceutical composition comprising an anti-sclerostin antibody as defined herein and a pharmaceutically acceptable carrier for use in the treatment of a bone-related disease, wherein a therapeutically effective amount of the anti-sclerostin antibody is administered each month for a period of at least 13 consecutive months. In some embodiments of the pharmaceutical compositions, the antibody comprises a VH comprising an HCDR1, an HCDR2, and an HCDR3 domain of the VH sequence of SEQ ID NO:70, and a VL comprising an LCDR1, an LCDR2, and an LCDR3 domain of the VL sequence of SEQ ID NO:81. Pharmaceutically acceptable carriers include a sterile aqueous solution.

### Certain Embodiments

In one embodiment, the present invention provides a method for treating OI in a human patient comprising administering to the human patient a therapeutically effective amount of 150-2500 mg of an anti-sclerostin antibody each month for a period of at least 13 consecutive months up to 18 years.

In a related embodiment, the present invention provides a method for treating OI in a human patient comprising administering to the human patient a therapeutically effective amount of 150-2500 mg of an anti-sclerostin antibody each month for a period of at least 30 consecutive months up to 18 years.

In another embodiment, the present invention provides a method for treating OI in a human patient aged 0-17 years comprising administering to the human patient a therapeutically effective amount of 20-40 mg/kg of an anti-sclerostin antibody each month for a period of at least 13 consecutive months up to 18 years.

In a related embodiment, the present invention provides a method for treating OI in a human patient aged 0-17 years comprising administering to the human patient a therapeutically effective amount of 20-40 mg/kg of an anti-sclerostin antibody each month for a period of at least 30 consecutive months up to 18 years.

In one embodiment, the present invention provides a method for treating OI in a human patient comprising administering to the human patient a therapeutically effective amount of 150-2500 mg of an anti-sclerostin antibody each month for a period of at least 13 consecutive months up to 18 years, wherein the anti-sclerostin antibody comprises a VH polypeptide sequence having the amino acid sequence of SEQ ID NO:70 and a VL polypeptide sequence having the amino acid sequence of SEQ ID NO:81.

In a related embodiment, the present invention provides a method for treating OI in a human patient comprising administering to the human patient a therapeutically effective amount of 150-2500 mg of an anti-sclerostin antibody each month for a period of at least 30 consecutive months up to 18 years, wherein the anti-sclerostin antibody comprises a VH polypeptide sequence having the amino acid sequence of SEQ ID NO:70 and a VL polypeptide sequence having the amino acid sequence of SEQ ID NO:81.

In another embodiment, the present invention provides a method for treating OI in a human patient aged 0-17 years comprising administering to the human patient a therapeutically effective amount of 20-40 mg/kg of an anti-sclerostin antibody each month for a period of at least 13 consecutive months up to 18 years, wherein the anti-sclerostin antibody comprises a VH polypeptide sequence having the amino acid sequence of SEQ ID NO:70 and a VL polypeptide sequence having the amino acid sequence of SEQ ID NO:81.

In a related embodiment, the present invention provides a method for treating OI in a human patient aged 0-17 years comprising administering to the human patient a therapeutically effective amount of 20-40 mg/kg of an anti-sclerostin antibody each month for a period of at least 30 consecutive months up to 18 years, wherein the anti-sclerostin antibody comprises a VH polypeptide sequence having the amino acid sequence of SEQ ID NO:70 and a VL polypeptide sequence having the amino acid sequence of SEQ ID NO:81.

In one embodiment, the present invention provides a method for treating OI in a human patient comprising administering to the human patient a therapeutically effective amount of 150-2500 mg of an anti-sclerostin antibody each month for a period of at least 13 consecutive months up to 18 years, wherein the anti-sclerostin antibody comprises a VH polypeptide sequence comprising the amino acid sequence of SEQ ID NO: 198 and a VL polypeptide sequence comprising the amino acid sequence of SEQ ID NO: 199.

In a related embodiment, the present invention provides a method for treating OI in a human patient comprising administering to the human patient a therapeutically effective amount of 150-2500 mg of an anti-sclerostin antibody each month for a period of at least 30 consecutive months up to 18 years, wherein the anti-sclerostin antibody comprises a VH polypeptide sequence comprising the amino acid sequence of SEQ ID NO: 198 and a VL polypeptide sequence comprising the amino acid sequence of SEQ ID NO: 199.

In another embodiment, the present invention provides a method for treating OI in a human patient aged 0-17 years comprising administering to the human patient a therapeutically effective amount of 20-40 mg/kg of an anti-sclerostin antibody each month for a period of at least 13 consecutive months up to 18 years, wherein the anti-sclerostin antibody comprises a VH polypeptide sequence comprising the amino acid sequence of SEQ ID NO: 198 and a VL polypeptide sequence comprising the amino acid sequence of SEQ ID NO: 199.

In a related embodiment, the present invention provides a method for treating OI in a human patient aged 0-17 years comprising administering to the human patient a therapeutically effective amount of 20-40 mg/kg of an anti-sclerostin antibody each month for a period of at least 30 consecutive months up to 18 years, wherein the anti-sclerostin antibody comprises a VH polypeptide sequence comprising the amino acid sequence of SEQ ID NO: 198 and a VL polypeptide sequence comprising the amino acid sequence of SEQ ID NO: 199.

In one embodiment, the present invention provides a method for treating OI in a human patient comprising administering to the human patient a therapeutically effective amount of 150-2500 mg of an anti-sclerostin antibody each month for a period of at least 13 consecutive months up to 18 years, wherein the anti-sclerostin antibody comprises a VH polypeptide sequence comprising the amino acid sequence of SEQ ID NO: 202 and a VL polypeptide sequence comprising the amino acid sequence of SEQ ID NO: 203.

In a related embodiment, the present invention provides a method for treating OI in a human patient comprising administering to the human patient a therapeutically effective amount of 150-2500 mg of an anti-sclerostin antibody each month for a period of at least 30 consecutive months up to 18 years, wherein the anti-sclerostin antibody comprises a VH polypeptide sequence comprising the amino acid sequence of SEQ ID NO: 202 and a VL polypeptide sequence comprising the amino acid sequence of SEQ ID NO: 203.

In another embodiment, the present invention provides a method for treating OI in a human patient aged 0-17 years comprising administering to the human patient a therapeutically effective amount of 20-40 mg/kg of an anti-sclerostin antibody each month for a period of at least 13 consecutive months up to 18 years, wherein the anti-sclerostin antibody comprises a VH polypeptide sequence comprising the amino acid sequence of SEQ ID NO: 202 and a VL polypeptide sequence comprising the amino acid sequence of SEQ ID NO: 203.

In a related embodiment, the present invention provides a method for treating OI in a human patient aged 0-17 years comprising administering to the human patient a therapeutically effective amount of 20-40 mg/kg of an anti-sclerostin antibody each month for a period of at least 30 consecutive months up to 18 years, wherein the anti-sclerostin antibody comprises a VH polypeptide sequence comprising the amino acid sequence of SEQ ID NO: 202 and a VL polypeptide sequence comprising the amino acid sequence of SEQ ID NO: 203.

### MODES FOR CARRYING OUT THE INVENTION

### Example 1

This example describes a 12-month, randomized, double-blind, Phase 2b clinical study including 60 adults diagnosed with type I, III or IV Osteogenesis Imperfecta (01) and a confirmed COL1A1/COL1A2 mutation who have fractured over the previous 5 years. Patients received each month either 8 mg/kg or 20 mg/kg doses of setrusumab for a period of 12 months. Setrusumab was administered intravenously by infusion. The study measured percentage change from baseline at 6 and 12 months for areal bone mineral density (BMD) at the lumbar spine, as measured by DXA. Procollagen I N-terminal propeptide (PINP), a biomarker of bone formation, and C-terminal telopeptide (CTX-1), a biomarker of bone resorption, were also measured at 1, 3, 6, 9, and 12 months.

Of the 60 patients randomized to the setrusumab treatment groups, 29 were treated with 8 mg/kg, administered monthly and 31 were treated with 20 mg/kg, administered monthly. Baseline patient characteristics (Table 2) and medical histories (Table 3) of these patients were comparable across treatment groups.

TABLE 2
Baseline Patient Characteristics by Treatment Group

| | Setrusumab 8 mg/kg | Setrusumab 20 mg/kg |
|---|---|---|
| | (n = 29) | (n = 31) |
| Age, years, mean (SD) | 40.4 (14.3) | 40.6 (13.7) |
| Sex, men / women | 9 / 20 | 14 / 17 |
| OI type, type I vs type | 18 / 11 | 18 / 13 |
| III/IV | | |
| Height, cm, mean (SD) | 150.6 (22.0) | 150.4 (17.4) |
| Weight, kg, mean (SD) | 65.1 (20.6) | 63.2 (18.9) |
| [min, max] | [19.9, 120.7] | [37.4, 109.5] |
| BMI, kg/m², mean (SD) | 28.3 (5.0) | 28.31 (10.5) |
| Race, n (%) | | |
| White | 27 (93.1) | 29 (93.5) |
| Black | 1 (3.4) | 2 (6.5) |

TABLE 3
Baseline Medical History by Treatment Group

| | Setrusumab 8 mg/kg | Setrusumab 20 mg/kg |
|---|---|---|
| | (n = 29) | (n = 31) |
| Prior bisphosphonate use, | 2 (6.9) | 6 (19.4) |
| n (%) | | |
| Fracture within the past 5 | 29 (100) | 31 (100) |
| years, n (%) | | |
| Medical/Physical Aids | 28 (96.6) | 28 (90.3) |
| History, n (%) | | |
| History of Physical Aids, n | 7 (24.1) | 13 (41.9) |
| (%) | | |
| Walking aid | 3 (10.3) | 8 (25.8) |
| Wheelchair | 3 (10.3) | 5 (16.1) |
| Hearing aids | 1 (3.4) | 2 (6.5) |
| Orthosis | 1 (3.4) | 1 (3.2) |
| Corrective lens | 0 | 1 (3.2) |
| Medical History, n (%) | | |
| Cardiovascular | 11 (37.9) | 11 (35.5) |
| disorders | | |
| Respiratory disorders | 6 (20.7) | 9 (29.0) |
| Neurological disorders | 6 (20.7) | 13 (41.9) |
| DXA T-score (Lumbar | −2.3 (1.1) | −2.6 (1.4) |
| Spine), mean (SD) | | |
| DXA T-score (Total Hip), | −1.5 (1.1) | −1.6 (1.2) |
| mean (SD) | | |
| DXA T-score (Femoral | −1.7 (0.9) | −1.7 (1.1) |
| Neck), mean (SD) | | |

### Results

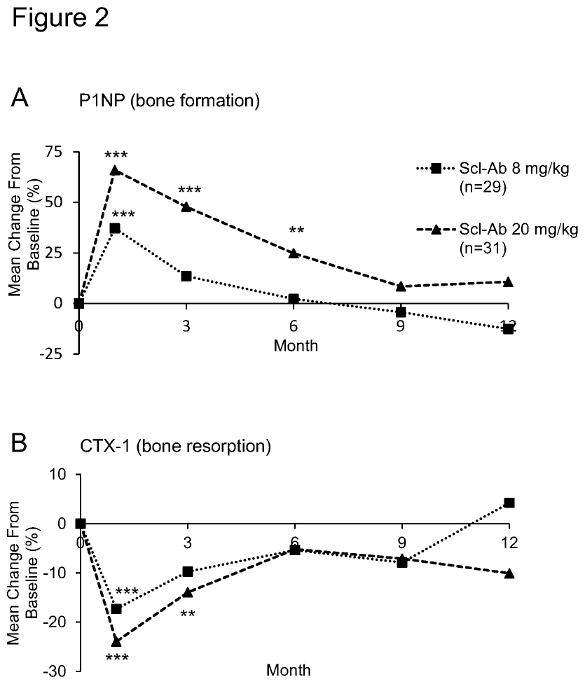

The assay results of the bone metabolism biomarkers for the study are presented in FIG. 2A and FIG. 2B. The results show dose-dependent improvements from baseline in serum biomarkers of bone turnover with setrusumab therapy. Surprisingly, both biomarkers showed a peak response in the first month, after which the response waned and within six to nine months of therapy starting the biomarker levels returned to levels that were not significantly different from baseline levels.

Levels of the bone formation biomarker (P1NP) were statistically significantly above baseline levels for the first six months of setrusumab therapy with 20 mg/kg monthly, and for the first month of therapy with 8 mg/kg monthly (FIG. 2A). Statistical significance was tested using an ANCOVA model with baseline values, treatment group and OI type as covariates (**p<0.01; ***p<0.001). With both doses of setrusumab, the P1NP levels peaked in the first month and then declined for the following 11 months of therapy.

Levels of the bone resorption marker (CTX-1) were significantly below baseline levels for the first three months of setrusumab therapy with 20 mg/kg monthly, and for the first month of therapy with 8 mg/kg monthly (FIG. 2B). Statistical significance was tested using an ANCOVA model with baseline values, treatment group and OI type as covariates (**p<0.01; ***p<0.001). With both doses of setrusumab, the CTX-1 levels reached their lowest in the first month and then increased again for the following 11 months of therapy.

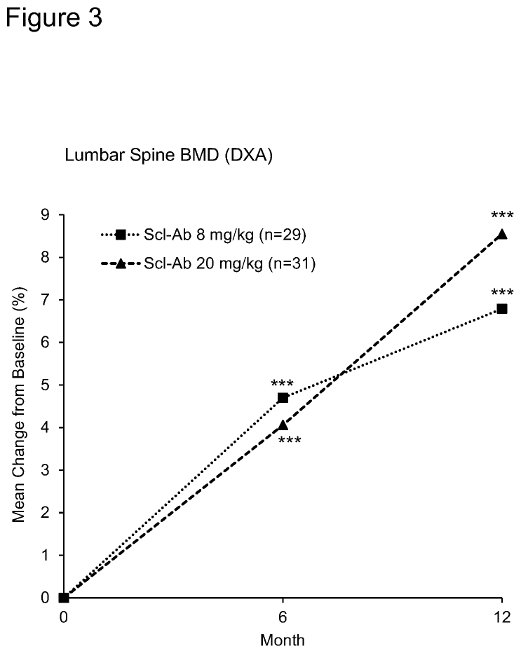

In contrast, FIG. 3 shows that improvements in lumbar spine BMD above baseline were continuous with setrusumab therapy. In the group receiving 20 mg/kg setrusumab monthly, lumbar spine BMD (as measured by DXA) increased by about 4% after six months, and 8.5% after 12 months. Similarly, in the group receiving 8 mg/kg setrusumab monthly, lumbar spine BMD (as measured by DXA) increased by about 4.7% after six months, and 6.8% after 12 months. These increases were statistically significant (***p<0.001) compared with baseline based on an ANCOVA model with baseline values, treatment group and OI type as covariates. So similar gains in BMD were observed for the first six months of monthly setrusumab therapy and the second six months despite biomarker attenuation early in therapy.

In addition, the study showed that the anti-sclerostin antibody was safe and well-tolerated in the adult OI patients. Few serious treatment emergent adverse events (TEAEs) were seen across the groups during the treatment period. Only two discontinuations were due to adverse events (AEs): neutropenia and headache. There were no clinically significant abnormalities of hematological, clinical chemistry, urinalysis, ECG or vital sign data compromising the patients' safety.

According to the clinical study results, setrusumab provides continuous increase in BMD in OI patients even though biomarkers appear to show a waning effect of setrusumab with long-term use. Surprisingly, these results show that the response analysed by biomarkers was distinct from the effect on BMD. Observed improvements in BMD were continuous, with comparable gains achieved in the first six months and second six months of treatment. In contrast, temporal changes were observed in biomarkers, with the biomarker response peaking in the first month of setrusumab therapy, and then rapidly waning even though therapy was continued. These results therefore show that setrusumab continues to have a clinically relevant effect increased BMD with long-term use, even though the response observed in levels of bone turnover biomarkers waned early in therapy. These surprising results led to the conclusion that setrusumab could be used for long-term (or chronic) therapy, and that a dosing holiday is not needed for setrusumab therapy.

### Example 2

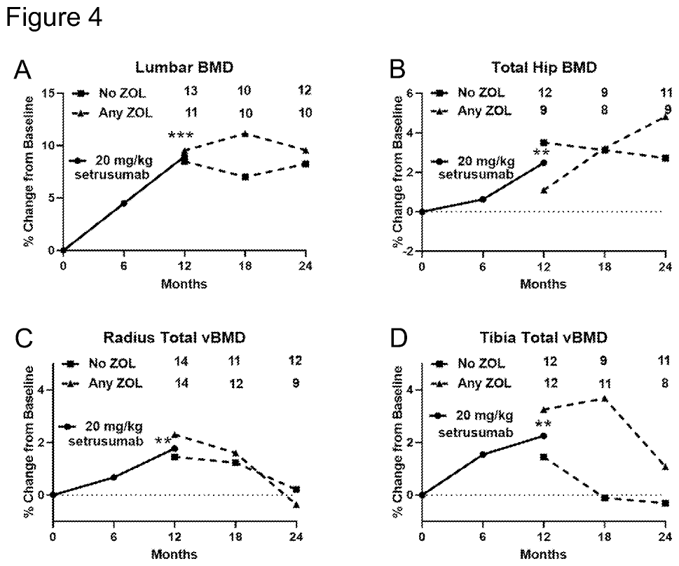

After 12 months of setrusumab therapy, setrusumab was discontinued and BMD was monitored through month 24. A subset of patients received zoledronic acid therapy (“Any ZOL”) at Month 12 and/or Month 18, and another subset received no zoledronic acid therapy (“No ZOL”) following setrusumab discontinuation. Lumbar BMD (FIG. 4A), Total Hip BMD (FIG. 4B), Radius Total volumetric BMD (FIG. 4C), and Tibia Total volumetric BMD (FIG. 4D) were evaluated over this 1-year period. Initial gains in BMD attributable to setrusumab therapy diminished over the 1-year period following discontinuation of setrusumab therapy. Greater losses in BMD were observed in the peripheral skeleton, while more moderate BMD losses were observed at the spine and hip. Administration of zoledronic acid (ZOL) prevented BMD loss at the spine and hip, but not in the distal radius and tibia.

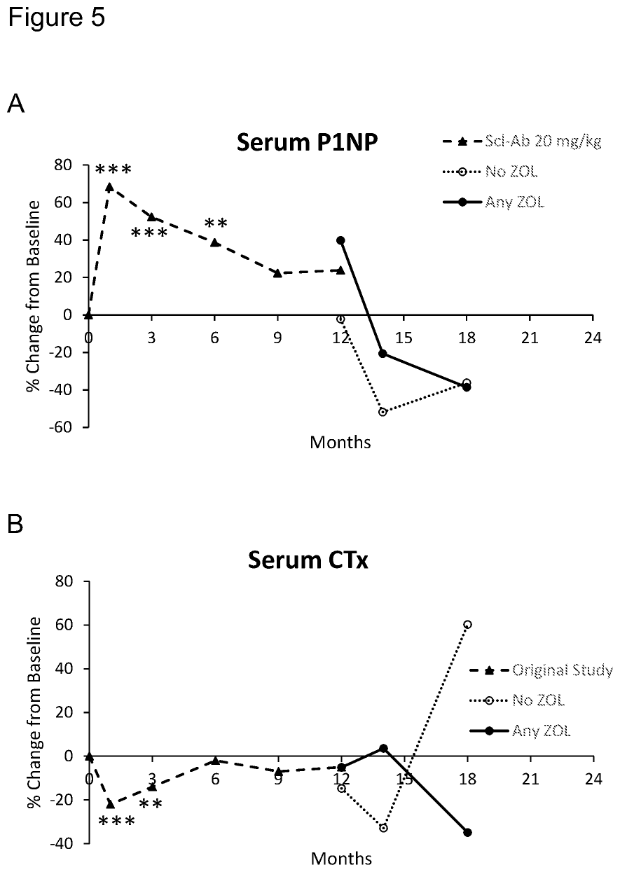

Bone turnover markers P1NP and CTx were evaluated in serum of patients throughout the course of the 12-month treatment with setrusumab (20 mg/kg) and following the treatment until month 18 (for a total of 6 months post-setrusumab treatment). Patients received zoledronic acid or none according to physician recommendation. Serum P1NP (indicative of bone formation) dropped in patients irrespective of treatment with zoledronic acid (ZOL) (FIG. 5A). On the other hand, serum CTx (indicative of bone resorption) increased in patients not receiving zoledronic acid.

Patients were also evaluated for adverse events in the follow-up period after the initial 12-month dosing period. Safety findings in the follow-up period were in line with expected outcomes, with fracture rates that varied but did not significantly exceed those during the initial 12-month treatment period. There were no major adverse cardiovascular events during the entire 2-year study.

These results show that not only do BMD gains resulting from setrusumab therapy continue well after the decline suggested by bone turnover biomarkers (Example 1), but also that antiresorptive therapies such as zoledronic acid alone are inadequate to maintain the early gains from setrusumab. Further, early peaks and valleys seen in bone turnover biomarkers with setrusumab treatment gradually even out over the course of treatment with setrusumab, but then switch from steady maintenance levels to rapid changes away from baseline once setrusumab is stopped. In the case of P1NP (bone formation), stopping setrusumab results in a decrease irrespective of zoledronic acid treatment. In the case of CTx (bone resorption), stopping setrusumab results in a rapid increase. Thus, BMD losses and changes in bone turnover biomarkers following discontinuation of setrusumab therapy support the need for continued treatment after 12 months.

The teachings of all patents, published applications and references cited herein are incorporated by reference in their entirety for all purposes. The patents and publications listed herein describe the general skill in the art. In the case of any conflict between a cited reference and this specification, the specification shall control. In describing embodiments of the present application, specific terminology is employed for the sake of clarity. However, the invention is not intended to be limited to the specific terminology so selected. Nothing in this specification should be considered as limiting the scope of the present invention. All examples presented are representative and non-limiting. The above-described embodiments may be modified or varied, without departing from the invention, as appreciated by those skilled in the art in light of the above teachings. It is therefore to be understood that, within the scope of the claims and their equivalents, the invention may be practiced otherwise than as specifically described.

What is claimed:

1. A method for treating osteogenesis imperfecta (OI) in a human patient comprising administering to the human patient a therapeutically effective amount of an anti-sclerostin antibody each month for a period of at least 13 consecutive months.

2. The method according to claim 1, wherein the anti-sclerostin antibody comprises:
(a) a heavy chain variable region (VH) polypeptide sequence having at least 90 percent sequence identity to the amino acid sequence of SEQ ID NO: 70; and/or
(b) a light chain variable region (VL) polypeptide sequence having at least 90 percent sequence identity to the amino acid sequence of SEQ ID NO: 81.

3. The method according to claim 2, wherein the anti-sclerostin antibody comprises a VH polypeptide sequence comprising the amino acid sequence set forth in SEQ ID NO: 70 and a VL polypeptide sequence comprising the amino acid sequence set forth in SEQ ID NO: 81.

4. The method according to claim 1, wherein the anti-sclerostin antibody comprises:
(a) an HCDR1 having the amino acid sequence of SEQ ID NO:4;
(b) an HCDR2 having the amino acid sequence of SEQ ID NO:15;
(c) an HCDR3 having the amino acid sequence of SEQ ID NO:26;
(d) an LCDR1 having the amino acid sequence of SEQ ID NO:37;
(e) an LCDR2 having the amino acid sequence of SEQ ID NO:48; and
(f) an LCDR3 having the amino acid sequence of SEQ ID NO:59.

5. The method according to claim 1, wherein the anti-sclerostin antibody comprises:
(a) an HCDR1 having the amino acid sequence of SEQ ID NO: 178;
(b) an HCDR2 having the amino acid sequence of SEQ ID NO: 179;
(c) an HCDR3 having the amino acid sequence of SEQ ID NO:26;
(d) an LCDR1 having the amino acid sequence of SEQ ID NO:37;
(e) an LCDR2 having the amino acid sequence of SEQ ID NO: 180; and
(f) an LCDR3 having the amino acid sequence of SEQ ID NO: 181.

6. The method according to claim 1, wherein the anti-sclerostin antibody comprises:
(a) an HCDR1 having the amino acid sequence of SEQ ID NO:4;
(b) an HCDR2 having the amino acid sequence of SEQ ID NO: 179;
(c) an HCDR3 having the amino acid sequence of SEQ ID NO:26;
(d) an LCDR1 having the amino acid sequence of SEQ ID NO:37;
(e) (e) an LCDR2 having the amino acid sequence of SEQ ID NO: 180; and
(f) (f) an LCDR3 having the amino acid sequence of SEQ ID NO: 181.

7. The method according to claim 1, wherein the anti-sclerostin antibody comprises:
(a) a VH polypeptide sequence having at least 90 percent sequence identity to the amino acid sequence of SEQ ID NO: 198; and/or
(b) a VL polypeptide sequence having at least 90 percent sequence identity to the amino acid sequence of SEQ ID NO: 199.

8. The method according to claim 7, wherein the anti-sclerostin antibody comprises a VH polypeptide sequence comprising the amino acid sequence of SEQ ID NO: 198 and a VL polypeptide sequence comprising the amino acid sequence of SEQ ID NO:
199.

9. The method according to claim 1, wherein the anti-sclerostin antibody comprises:
(a) a VH polypeptide sequence having at least 90 percent sequence identity to the amino acid sequence of SEQ ID NO: 202; and/or
(b) a VL polypeptide sequence having at least 90 percent sequence identity to the amino acid sequence of SEQ ID NO: 203.

10. The method according to claim 9, wherein the anti-sclerostin antibody comprises a VH polypeptide sequence comprising the amino acid sequence of SEQ ID NO: 202 and a VL polypeptide sequence comprising the amino acid sequence of SEQ ID NO: 203.

11. The method according to claim 1, wherein the anti-sclerostin antibody is setrusumab.

12. The method according to claim 1, wherein the anti-sclerostin antibody is romosozumab.

13. The method according to claim 1, wherein the anti-sclerostin antibody is blosozumab.

14. The method according to any one of the preceding claims, wherein the therapeutically effective amount of anti-sclerostin antibody is administered monthly for a period of at least 18 consecutive months.

15. The method according to any one of the preceding claims, wherein the therapeutically effective amount of anti-sclerostin antibody is administered monthly for a period of at least 24 consecutive months.

16. The method according to any one of the preceding claims, wherein the therapeutically effective amount of anti-sclerostin antibody is administered monthly for a period of at least 30 consecutive months.

17. The method according to any one of the preceding claims, wherein the therapeutically effective amount of anti-sclerostin antibody is administered monthly for a period of at least 36 consecutive months.

18. The method according to any one of the preceding claims, wherein the anti-sclerostin antibody is administered monthly for a period of up to 18 years.

19. The method according to any one of the preceding claims, wherein administering the therapeutically effective amount of the anti-sclerostin antibody increases trabecular bone mineral density (BMD) after 12 months treatment of the human patient.

20. The method according to any one of the preceding claims, wherein administering the therapeutically effective amount of the anti-sclerostin antibody increases bone mineral density (BMD) of lumbar spine by 5% or more after 12 months treatment of the human patient.

21. The method according to any one of the preceding claims, wherein the OI is type I OI, type III 01 or type IV OI.

22. The method according to any one of the preceding claims, wherein the human patient has one or more mutations in the COL1A1 and/or COL1A2 genes.

23. The method according to any one of the preceding claims, wherein the human patient is aged 0-17 years.

24. The method according to any one of the preceding claims, wherein the human patient is a child aged 0-17 years and wherein the anti-sclerostin antibody is administered monthly at a dose of 20-50 mg/kg.

25. The anti-sclerostin antibody as defined in any one of claims 1-13, an anti-sclerostin antibody that cross-blocks the antibody as defined in any one of claims 1-13, or an antisclerostin antibody that binds to the same epitope as the antibody as defined in any one of claims 1-13, for use in a method of treatment according to any one of claims 1-24.

26. A method of treatment according to any of claims 1-24, wherein the anti-sclerostin antibody cross-blocks the antibody as defined in any one of claims 1-13 or binds to the same epitope as the antibody as defined in any one of claims 1-13.

27. A method for treating osteogenesis imperfecta (OI) in a human patient comprising administering to the human patient a therapeutically effective amount of an anti-sclerostin antibody, wherein the method comprises administering a first dosage monthly during an initial dosing period of 12 months or greater followed by regular dosing at a maintenance dosage.

28. The method according to claim 27, wherein the first dosage comprises 20-50 mg/kg of the anti-sclerostin antibody administered monthly.

29. The method according to any of claims 27-28, wherein the first dosage comprises 20-40 mg/kg of the anti-sclerostin antibody administered monthly.

30. The method according to any of claims 27-29, wherein the first dosage comprises 20 mg/kg of the anti-sclerostin antibody administered monthly.

31. The method according to any of claims 27-30, wherein the maintenance dosage comprises administration of the anti-sclerostin antibody monthly, every 2 months, every 3, months, every 4 months, every 6 months, or every 12 months.

32. The method according to any of claims 27-31, wherein the maintenance dosage comprises administration of an equal or reduced amount of the anti-sclerostin antibody relative to the amount administered during the initial dosing period.

33. The method according to any of claims 27-32, wherein the maintenance dosage comprises 20 mg/kg or less of the anti-sclerostin antibody.

34. The method according to any of claims 27-33, wherein the anti-sclerostin antibody is administered monthly at 20 mg/kg for a period of 12 months or longer during the initial dosing period, then at a maintenance dosage of 20 mg/kg or less every 2 months.

35. The method according to any of claims 27-34, where in the anti-sclerostin antibody is administered monthly at 20 mg/kg for a period of 12 months or longer during the initial dosing period, then at a maintenance dosage of 20 mg/kg every 2 months.

36. The method according to any of claims 27-34, wherein the anti-sclerostin antibody is administered monthly at 20 mg/kg for a period of 12 months during the initial dosing period, then at a maintenance dosage of less than 20 mg/kg every month.

37. The method according to any of claims 27-36, wherein the maintenance dosage is administered for a period of up to 18 years.

38. A method of treatment substantially as described herein or an anti-sclerostin antibody for use in a method of treatment substantially as described here.
